SEQUENCE LISTING (110 sequences)

SEQ ID NO: 1 - protein, 412 aa, Homo sapiens
  Cathepsin D precursor; Accession NO as of 06 Dec 2002 P07339
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 2 - protein, 414 aa, Homo sapiens
  Isocitrate dehydrogenase [NADP] cytoplasmic; Accession NO as of 06 Dec 2002 O75874
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 3 - protein, 782 aa, Homo sapiens
  Gelsolin precursor, plasma; Accession NO as of 06 Dec 2002 P06396
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 4 - protein, 764 aa, Homo sapiens
  Complement factor B precursor; Accession NO as of 06 Dec 2002 P00751
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 5 - protein, 911 aa, Homo sapiens
  Alpha-actinin 4; Accession NO O43707
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL

SEQ ID NO: 6 - protein, 683 aa, Homo sapiens
  Transforming growth factor-beta induced protein IG-H3 precursor; Accession NO as of 06 Dec 2002 Q15582
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 7 - protein, 892 aa, Homo sapiens [sequence omitted]

SEQ ID NO: 8 - protein, 448 aa, Homo sapiens
  Tubulin alpha-4 chain; Accession NO P05215
MRECISVHVGQAGVQMGNACWELYCLEHGIQPDGQMPSDKTIGGGDDSFTTFFCETGAGKHVPRAVFVDLEPTVIDEIRNGPYRQLFHPEQLITGKEDAANNYARGHYTIGKEIIDPVLDRIRKLSDQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIAAIKTKRSIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGIDSYEDEDEGEE

SEQ ID NO: 9 - protein, 2647 aa, Homo sapiens
  Filamin A; Accession NO P21333
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGHEHIPGSPYRVVVP

SEQ ID NO: 10 - protein, 199 aa, Homo sapiens
  Transgelin 2; Accession NO as of 06 Dec 2002 P37802
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 11 - protein, 248 aa, Homo sapiens
  Tropomyosin alpha 4 chain; Accession NO P07226
MAGLNSLEAVKRKIQALQQQADEAEDRAQGLQRELDGERERREKAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 12 - protein, 793 aa, Homo sapiens
  Caldesmon; Accession NO as of 06 Dec 2002 Q05682
MDDFERRRELRRQKREEMRLEAERIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQVEVMVEEKTTESQEETVVMSLKNGQISSEEPKQEEEREQGSDEISHHEKMEEEDKERAEAERARLEAEERERIKAEQDKKIADERARIEAEEKAAAQERERREAEERERMREEEKRAAEERQRIKEEEKRAAEERQRIKEEEKRAAEERQRIKEEEKRAAEERQRARAEEEEKAKVEEQKRNKQLEEKKRAMQETKIKGEKVEQKIEGKWVNEKKAQEDKLQTAVLKKQGEEKGTKVQAKREKLQEDKPTFKKEEIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 13 - protein, 458 aa, Homo sapiens [sequence omitted]

SEQ ID NO: 14 - protein, 408 aa, Homo sapiens
  Aminoacylase-1; Accession NO as of 06 Dec 2002 Q03154
MTSKGPEEEHPSVTLFRQYLRIRTVQPKPDYGAAVAFFEETARQLGLGCQKVEVAPGYVVTVLTWPGTNPTLSSILLNSHTDVVPVFKEHWSHDPFEAFKDSEGYIYARGAQDMKCVSIQYLEAVRRLKVEGHRFPRTIHMTFVPDEEVGGHQGMELFVQRPEFHALRAGFALDEGIANPTDAFTVFYSERSPWWVRVTSTGRPGHASRFMEDTAAEKLHKVVNSILAFREKEWQRLQSNPHLKEGSVTSVNLTKLEGGVAYNVIPATMSASFDFRVAPDVDFKAFEEQLQSWCQAAGEGVTLEFAQKWMHPQVTPTDDSNPWWAAFSRVCKDMNLTLEPEIMPAATDNRYIRAVGVPALGFSPMNRTPVLLHDHDERLHEAVFLRGVDIYTRLLPALASVPALPSDS

SEQ ID NO: 15 - protein, 277 aa, Homo sapiens
  F-actin capping protein beta subunit; Accession NO as of 06 Dec 2002 P47756
MSDQQLDCALDLMRRLPPQQIEKNLSDLIDLVPSLCEDLLSSVDQPLKIARDKVVGKDYLLCDYNRDGDSYRSPWSNKYDPPLEDGAMPSARLRKLEVEANNAFDQYRDLYFEGGVSSVYLWDLDHGFAGVILIKKAGDGSKKIKGCWDSIHVVEVQEKSSGRTAHYKLTSTVMLWLQTNKSGSGTMNLGGSLTRQMEKDETVSDCSPHIANIGRLVEDMENKIRSTLNEIYFGKTKDIVNGLRSIDAIPDNQKFKQLQRELSQVLTQRQIYIQPDN

SEQ ID NO: 16 - protein, 289 aa, Homo sapiens
  Inorganic pyrophosphatase; Accession NO as of 06 Dec 2002 Q15181
MSGFSTEERAAPFSLEYRVFLKNEKGQYISPFHDIPIYADKDVFHMVVEVPRWSNAKMEIATKDPLNPIKQDVKKGKLRYVANLFPYKGYIWNYGAIPQTWEDPGHNDKHTGCCGDNDPIDVCEIGSKVCARGEIIGVKVLGILAMIDEGETDWKVIAINVDDPDAANYNDINDVKRLKPGYLEATVDWFRRYKVPDGKPENEFAFNAEFKDKDFAIDIIKSTHDHWKALVTKKTNGKGISCMNTTLSESPFKCDPDAARAIVDALPPPCESACTVPTDVDKWFHHQKN

SEQ ID NO: 17 - protein, 250 aa, Homo sapiens
  Galectin-3 (Galactose-specific lectin 3); Accession NO as of 06 Dec 2002 P17931
MADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 18 - protein, 347 aa, Homo sapiens [sequence omitted]

SEQ ID NO: 19 - protein, 339 aa, Homo sapiens
  Annexin II; Accession NO as of 06 Dec 2002 P07355
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 20 - protein, 418 aa, Homo sapiens
  Collagen-binding protein 2 precursor; Accession NO as of 06 Dec 2002 P50454
MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 21 - protein, 166 aa, Homo sapiens
  Cofilin, non-muscle isoform; Accession NO as of 08 ec 2002 P23528
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 22 - protein, 165 aa, Homo sapiens
  Peptidyl-prolyl cis-trans isomerase A; Accession NO as of 09 Dec 2002 P05092
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 23 - protein, 638 aa, Homo sapiens
  Dynein intermediate chain 2, cytosolic; Accession NO as of 09 Dec 2002 Q13409
MSDKSELKAELERKKQRLAQIREEKKRKEEERKKKETDQKKEAVAPVQEESDLEKKRREAEALLQSMGLTPESPIVFSEYWVPPPMSPSSKSVSTPSEAGSQDSGDGAVGSRTLHWDTDPSVLQLHSDSDLGRGPIKLGMAKITQVDFPPREIVTYTKETQTPVMAQPKEDEEEDDDVVAPKPPIEPEEEKTLKKDEENDSKAPPHELTEEEKQQILHSEEFLSFFDHSTRIVERALSEQINIFFDYSGRDLEDKEGEIQAGAKLSLNRQFFDERWSKHRVVSCLDWSSQYPELLVASYNNNEDAPHEPDGVALVWNMKYKKTTPEYVFHCQSAVMSATFAKFHPNLVVGGTYSGQIVLWDNRSNKRTPVQRTPLSAAAHTHPVYCVNVVGTQNAHNLISISTDGKICSWSLDMLSHPQDSMELVHKQSKAVAVTSMSFPVGDVNNFVVGSEEGSVYTACRHGSKAGISEMFEGHQGPITGIHCHAAVGAVDFSHLFVTSSFDWTVKLWTTKNNKPLYSFEDNADYVYDVMWSPTHPALFACVDGMGRLDLWNLNNDTEVPTASISVEGNPALNRVRWTHSGREIAVGDSEGQIVIYDVGEQIAVPRNDEWARFGRTLAEINANRADAEEEAATRIPA

SEQ ID NO: 24 - protein, 328 aa, Homo sapiens
  Delta3,5-delta2,4-dienoyl-CoA isomerase, mitochondrial precursor; Accession NO as of 09 Dec 2002 Q13011
MAAGIVASRRLRDLLTRRLTGSNYPGLSISLRLTGSSAQEEASGVALGEAPDHSYESLRVTSAQKHVLHVQLNRPNKRNAMNKVFWREMVECFNKISRDADCRAVVISGAGKMFTAGIDLMDMASDILQPKGDDVARISWYLRDIITRYQETFNVIERCPKPVIAAVHGGCIGGGVDLVTACDIRYCAQDAFFQVKEVDVGLAADVGTLERLPKVIGNQSLVNELAFTAHKMMADEALDSGLVSRVFPDKEVMLDAALPLAPEISSKTTVLVQSTKVNLLYSRDHSVAESLNYVASWNMSMLQTQDLVKSVQPTTENKELKTVTFSKL

SEQ ID NO: 25 - protein, 1657 aa, Homo sapiens
  Ras GTPase-activating-like protein IQGAP1; Accession NO as of 09 Dec 2002 P46940
MSAADEVDGLGVARPHYGSVLDNERLTAEEMDERRRQNVAYEYLCHLEEAKRWMEACLGEDLPPTTELEEGLRNGVYLAKLGNFFSPKVVSLKKIYDREQTRYKATGLHFRHTDNVIQWLNAMDEIGLPKIFYPETTDIYDRKNMPRCIYCIHALSLYLFKLGLAPQIQDLYGKVDFTEEEINNMKTELEKYGIQMPAFSKIGGILANELSVDEAALHAAVIAINEAIDRRIPADTFAALKNPNAMLVNLEEPLASTYQDILYQAKQDKMTNAKNRTENSERERDVYEELLTQAEIQGNINKVNTFSALANIDLALEQGDALALFRALQSPALGLRGLQQQNSDWYLKQLLSDKQQKRQSGQTDPLQKEELQSGVDAANSAAQQYQRRLAAVALINAAIQKGVAEKTVLELMNPEAQLPQVYPFAADLYQKELATLQRQSPEHNLTHPELSVAVEMLSSVALINRALESGDVNTVWKQLSSSVTGLTNIEEENCQRYLDELMKLKAQAHAENNEFITWNDIQACVDHVNLVVQEEHERILAIGLINEALDEGDAQKTLQALQIPAAKLEGVLAEVAQHYQDTLIRAKREKAQEIQDESAVLWLDEIQGGIWQSNKDTQEAQKFALGIFAINEAVESGDVGKTLSALRSPDVGLYGVIPECGETYHSDLAEAKKKKLAVGDNNSKWVKHWVKGGYYYYHNLETQEGGWDEPPNFVQNSMQLSREEIQSSISGVTAAYNREQLWLANEGLITRLQARCRGYLVRQEFRSRMNFLKKQIPAITCIQSQWRGYKQKKAYQDRLAYLRSHKDEVVKIQSLARMHQARKRYRDRLQYFRDHINDIIKIQAFIRANKARDDYKTLINAEDPPMVVVRKFVHLLDQSDQDFQEELDLMKMREEVITLIRSNQQLENDLNLMDIKIGLLVKNKITLQDVVSHSKKLTKKNKEQLSDMMMINKQKGGLKALSKEKREKLEAYQHLFYLLQTNPTYLAKLIFQMPQNKSTKFMDSVIFTLYNYASNQREEYLLLRLFKTALQEEIKSKVDQIQEIVTGNPTVIKMVVSFNRGARGQNALRQILAPVVKEIMDDKSLNIKTDPVDIYKSWVNQMESQTGEASKLPYDVTPEQALAHEEVKTRLDSSIRNMRAVTDKFLSAIVSSVDKIPYGMRFIAKVLKDSLHEKFPDAGEDELLKIIGNLLYYRYMNPAIVAPDAFDIIDLSAGGQLTTDQRRNLGSIAKMLQHAASNKMFLGDNAHLSIINEYLSQSYQKFRRFFQTACDVPELQDKFNVDEYSDLVTLTKPVIYISIGEIINTHTLLLDHQDAIAPEHNDPIHELLDDLGEVPTIESLIGESSGNLNDPNKEALAKTEVSLTLTNKFDVPGDENAEMDARTILLNTKRLIVDVIRFQPGETLTEILETPATSEQEAEHQRAMQRRAIRDAKTPDKMKKSKSVKEDSNLTLQEKKEKIQTGLKKLTELGTVDPKNKYQELINDIARDIRNQRRYRQRRKAELVKLQQTYAALNSKATFYGEQVDYYKSYIKTCLDNLASKGKVSKKPREMKGKKSKKISLKYTAARLHEKGVLLEIEDLQVNQFKNVIFEISPTEEVGDFEVKAKFMGVQMETFMLHYQDLLQLQYEGVAVMKLFDRAKVNVNLLIFLLNKKFYGK

SEQ ID NO: 26 - protein, 627 aa, Homo sapiens
  L-plastin (Lymphocyte cytosolic protein 1); Accession NO as of 09 Dec 2002 P13796
MARGSVSDEEMMELREAFAKVDTDGNGYISFNELNDLFKAACLPLPGYRVREITENLMATGDLDQDGRISFDEFIKIFHGLKSTDVAKTFRKAINKKEGICAIGGTSEQSSVGTQHSYSEEEKYAFVNWINKALENDPDCRHVIPMNPNTNDLFNAVGDGIVLCKMINLSVPDTIDERTINKKKLTPFTIQENLNLALNSASAIGCHVVNIGAEDLKEGKPYLVLGLLWQVIKIGLFADIELSRNEALIALLREGESLEDLMKLSPEELLLRWANYHLENAGCNKIGNFSTDIKDSKAYYHLLEQVAPKGDEEGVPAVVIDMSGLREKDDIQRAECMLQQAERLGCRQFVTATDVVRGNPKLNLAFIANLFNRYPALHKPENQDIDWGALEGETREERTFRNWMNSLGVNPRVNHLYSDLSDALVIFQLYEKIKVPVDWNRVNKPPYPKLGGNMKKLENCNYAVELGKNQAKFSLVGIGGQDLNEGNRTLTLALIWQLMRRYTLNILEEIGGGQKVNDDIIVNWVNETLREAEKSSSISSFKDPKISTSLPVLDLIDAIQPGSINYDLLKTENLNDDEKLNNAKYAISMARKIGARVYALPEDLVEVNPKMVMTVFACLMGKGMKRV

SEQ ID NO: 27 - protein, 216 aa, Homo sapiens
  GTP-binding nuclear protein RAN; Accession NO as of 09 Dec 2002 P17080
MAAQGEPQVQFKLVLVGDGGTGKTTFVKRHLTGEFEKKYVATLGVEVHPLVFHTNRGPIKFNVWDTAGQEKFGGLRDGYYIQAQCAIIMFDVTSRVTYKNVPNWHRDLVRVCENIPIVLCGNKVDIKDRKVKAKSIVFHRKKNLQYYDISAKSNYNFEKPFLWLARKLIGDPNLEFVAMPALAPPEVVMDPALAAQYEHDLEVAQTTALPDEDDDL

SEQ ID NO: 28 - protein, 463 aa, Homo sapiens
  Heterogeneous nuclear ribonucleoprotein K; Accession NO as of 09 Dec 2002 Q07244
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 29 - protein, 172 aa, Homo sapiens
  Translationally controlled tumor protein (TCTP); Accession NO as of 09 Dec 2002 P13693
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC

SEQ ID NO: 30 - protein, 284 aa, Homo sapiens
  Tropomyosin 1 alpha chain; Accession NO as of 06 Dec 2002 P09493
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDRYEEEIKVLSDKLKEAETRAEFAERSVTKLEKSIDDLEDELYAQKLKYKAISEELDHALNDMTSI

SEQ ID NO: 31 - protein, 482 aa, Homo sapiens [sequence omitted]

SEQ ID NO: 32 - protein, 488 aa, Homo sapiens
  Cytosol aminopeptidase; Accession NO as of 09 Dec 2002 P28838
MTKGLVLGIYSKEKEDDVPQFTSAGENFDKLLAGKLRETLNISGPPLKAGKTRTFYGLHQDFPSVVLVGLGKKAAGIDEQENWHEGKENIRAAVAAGCRQIQDLELSSVEVDPCGDAQAAAEGAVLGLYEYDDLKQKKKMAVSAKLYGSGDQEAWQKGVLFASGQNLARQLMETPANEMTPTRFAEIIEKNLKSASSKTEVHIRPKSWIEEQAMGSFLSVAKGSDEPPVFLEIHYKGSPNANEPPLVFVGKGITFDSGGISIKASANMDLMRADMGGAATICSAIVSAAKLNLPINIIGLAPLCENMPSGKANKPGDVVRAKNGKTIQVDNTDAEGRLILADALCYAHTFNPKVILNAATLTGAMDVALGSGATGVFTNSSWLWNKLFEASIETGDRVWRMPLFEHYTRQVVDCQLADVNNIGKYRSAGACTAAAFLKEFVTHPKWAHLDIAGVMTNKDEVPYLRKGMTGRPTRTLIEFLLRFSQDNA

SEQ ID NO: 33 - protein, 400 aa, Homo sapiens
  Keratin, type I cytoskeletal 19; Accession NO as of 09 Dec 2002  P08727
MTSYSYRQSSATSSFGGLGGGSVRFGPGVAFRAPSIHGGSGGRGVSVSSARFVSSSSSGGYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLADVRADSERQNQEYQRLMDIKSRLEQEIATYRSLLEGQEDHYNNLSASKVL

SEQ ID NO: 34 - protein, 325 aa, Homo sapiens
  Alcohol dehydrogenase [NADP+]; Accession NO as of 09 Dec 2002 P14550
MAASCVLLHTGQKMPLIGLGTWKSEPGQVKAAVKYALSVGYRHIDCAAIYGNEPEIGEALKEDVGPGKAVPREELFVTSKLWNTKHHPEDVEPALRKTLADLQLEYLDLYLMHWPYAFERGDNPFPKNADGTICYDSTHYKETWKALEALVAKGLVQALGLSNFNSRQIDDILSVASVRPAVLQVECHPYLAQNELIAHCQARGLEVTAYSPLGSSDRAWRDPDEPVLLEEPVVLALAEKYGRSPAQILLRWQVQRKVICIPKSITPSRILQNIKVFDFTFSPEEMKQLNALNKNWRYIVPMLTVDGKRVPRDAGHPLYPFNDPY

SEQ ID NO: 35 - protein, 270 aa, Homo sapiens
  Elastase IIIA precursor; Accession NO as of 09 Dec 2002 P09093
MMLRLLSSLLLVAVASGYGPPSSHSSSRVVHGEDAVPYSWPWQVSLQYEKSGSFYHTCGGSLIAPDWVVTAGHCISRDLTYQVVLGEYNLAVKEGPEQVIPINSEELFVHPLWNRSCVACGNDIALIKLSRSAQLGDAVQLASLPPAGDILPNKTPCYITGWGRLYTNGPLPDKLQQARLPVVDYKHCSRWNWWGSTVKKTMVCAGGYIRSGCNGDSGGPLNCPTEDGGWQVHGVTSFVSGFGCNFIWKPTVFTRVSAFIDWIEETIASH

SEQ ID NO: 36 - protein, 509 aa, Homo sapiens
  Dihydrolipoamide dehydrogenase, mitochondrial precursor; Accession NO as of 09 Dec 2002 P09622
MQSWSRVYCSLAKRGHFNRISHGLQGLSAVPLRTYADQPIDADVTVIGSGPGGYVAAIKAAQLGFKTVCIEKNETLGGTCLNVGCIPSKALLNNSHYYHMAHGTDFASRGIEMSEVRLNLDKMMEQKSTAVKALTGGIAHLFKQNKVVHVNGYGKITGKNQVTATKADGGTQVIDTKNILIATGSEVTPFPGITIDEDTIVSSTGALSLKKVPEKMVVIGAGVIGVELGSVWQRLGADVTAVEFLGHVGGVGIDMEISKNFQRILQKQGFKFKLNTKVTGATKKSDGKIDVSIEAASGGKAEVITCDVLLVCIGRRPFTKNLGLEELGIELDPRGRIPVNTRFQTKIPNIYAIGDVVAGPMLAHKAEDEGIICVEGMAGGAVHIDYNCVPSVIYTHPEVAWVGKSEEQLKEEGIEYKVGKFPFAANSRAKTNADTDGMVKILGQKSTDRVLGAHILGPGAGEMVNEAALALEYGASCEDIARVCHAHPTLSEAFREANLAASFGKSINF

SEQ ID NO: 37 - protein, 290 aa, Homo sapiens
  Enoyl-CoA hydratase, mitochondrial precursor Accession NO as of 09 Dec 2002 P30084
MAALRVLLSCARGPLRPPVRCPAWRPFASGANFEYIIAEKRGKNNTVGLIQLNRPKALNALCDGLIDELNQALKIFEEDPAVGAIVLTGGDKAFAAGADIKEMQNLSFQDCYSSKFLKHWDHLTQVKKPVIAAVNGYAFGGGCELAMMCDIIYAGEKAQFAQPEILIGTIPGAGGTQRLTRAVGKSLAMEMVLTGDRISAQDAKQAGLVSKICPVETLVEEAIQCAEKIASNSKIVVAMAKESVNAAFEMTLTEGSKLEKKLFYSTFATDDRKEGMTAFVEKRKANFKDQ

SEQ ID NO: 38 - protein, 160 aa, Homo sapiens
  Heat-shock 20 kDa like-protein p20; Accession NO as of 09 Dec 2002 O14558
MEIPVPVQPSWLRRASAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAK

SEQ ID NO: 39 - protein, 151 aa, Homo sapiens
  Myosin light chain alkali, non-muscle isoform; Accession NO as of 09 Dec 2002 P16475
MCDFTEDQTAEFKEAFQLFDRTGDGKILYSQCGDVMRALGQNPTNAEVLKVLGNPKSDEMNVKVLDFEHFLPMLQTVAKNKDQGTYEDYVEGLRVFDKEGNGTVMGAEIRHVLVTLGEKMTEEEVEMLVAGHEDSNGCINYEAFVRHILSG

SEQ ID NO: 40 - protein, 592 aa, Homo sapiens
  Calnexin precursor; Accession NO as of 09 Dec 2002 P27824
MEGKWLLCMLLVLGTAIVEAHDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 41 - protein, 282 aa, Homo sapiens
  Complement component 1; Accession NO as of 09 Dec 2002 Q07021
MLPLLRCVPRVLGSSVAGLRAAAPASPFRQLLQPAPRLCTRPFGLLSVRAGSERRPGLLRPRGPCACGCGCGSLHTDGDKAFVDFLSDEIKEERKIQKHKTLPKMSGGWELELNGTEAKLVRKVAGEKITVTFNINNSIPPTFDGEEEPSQGQKVEEQEPELTSTPNFVVEVIKNDDGKKALVLDCHYPEDEVGQEDEAESDIFSIREVSFQSTGESEWKDTNYTLNTDSLDWALYDHLMDFLADRGVDNTFADELVELSTALEHQEYITFLEDLKSFVKSQ

SEQ ID NO: 42 - protein, 727 aa, Homo sapiens
  NADH-ubiquinone oxidoreductase 75 kDa subunit, Mitochondrial Precursor; Accession NO as of 09 Dec 2002 P28331
MLRIPVRRALVGLSKSPKGCVRTTATAASNLIEVFVDGQSVMVEPGTTVLQACEKVGMQIPRFCYHERLSVAGNCRMCLVEIEKAPKVVAACAMPVMKGWNILTNSEKSKKAREGVMEFLLANHPLDCPICDQGGECDLQDQSMMFGNDRSRFLEGKRAVEDKNIGPLVKTIMTRCIQCTRCIRFASEIAGVDDLGTTGRGNDMQVGTYIEKMFMSELSGNIIDICPVGALTSKPYAFTARPWETRKTESIDVMDAVGSNIVVSTRTGEVMRILPRMHEDINEEWISDKTRFAYDGLKRQRLTEPMVRNEKGLLTYTSWEDALSRVAGMLQSFQGKDVAAIAGGLVDAEALVALKDLLNRVDSDTLCTEEVFPTAGAGTDLRSNYLLNTTIAGVEEADVVLLVGTNPRFEAPLFNAWIRKSWLHNDLKVALIGSPVDLTYTYDHLGDSPKILQDIASGSHPFSQVLKEAKKPMVVLGSSALQRNDGAAILAAVSSIAQKIRMTSGVTGDWKVMNILHRIASQVAALDLGYKPGVEAIRKNPPKVLFLLGADGGCITRQDLPKDCFIIYQGHHGDVGAPIADVILPGAAYTEKSATYVNTEGRAQQTKVAVTPPGLAREDWKIIRALSEIAGMTLPYDTLDQVRNRLEEFSPNLVRYDDIEGANYFQQANELSKLVNQQLLADPLVPPQLTLKDFYMTDSISRASQTMAKCVKAVTEGAQAVEEPSIC

SEQ ID NO: 43 - protein, 491 aa, Homo sapiens [sequence omitted]

SEQ ID NO: 44 - protein, 135 aa, Homo sapiens
  Retinol-binding protein I, cellular; Accession NO as of 09 Dec 2002 P09455
MPVDFTGYWKMLVNENFEEYLRALDVNVALRKIANLLKPDKEIVQDGDHMIIRTLSTFRNYIMDFQVGKEFEEDLTGIDDRKCMTTVSWDGDKLQCVQKGEKEGRGWTQWIEGDELHLEMRVEGVVCKQVFKKVQ

SEQ ID NO: 45 - protein, 544 aa, Homo sapiens
  T-complex protein 1, gamma subunit; Accession NO as of 09 Dec 2002 P49368
MGHRPVLVLSQNTKRESGRKVQSGNINAAKTIADIIRTCLGPKSMMKMLLDPMGGIVMTNDGNAILREIQVQHPAAKSMIEISRTQDEEVGDGTTSVIILAGEMLSVAEHFLEQQMHPTVVISAYRKALDDMISTLKKISIPVDISDSDMMLNIINSSITTKAISRWSSLACNIALDAVKMVQFEENGRKEIDIKKYARVEKIPGGIIEDSCVLRGVMINKDVTHPRMRRYIKNPRIVLLDSSLEYKKGESQTDIEITREEDFTRILQMEEEYIQQLCEDIIQLKPDVVITEKGISDLAQHYLMRANITAIRRVRKTDNNRIARACGARIVSRPEELREDDVGTGAGLLEIKKIGDEYFTFITDCKDPKACTILLRGASKEILSEVERNLQDAMQVCRNVLLDPQLVPGGGASEMAVAHALTEKSKAMTGVEQWPYRAVAQALEVIPRTLIQNCGASTIRLLTSLRAKHTQENCETWGVNGETGTLVDMKELGIWEPLAVKLQTYKTAVETAVLLLRIDDIVSGHKKKGDDQSRQGGAPDAGQE

SEQ ID NO: 46 - protein, 461 aa, Homo sapiens [sequence omitted]

SEQ ID NO: 47 - protein, 317 aa, Homo sapiens [sequence omitted]

SEQ ID NO: 48 - protein, 172 aa, Homo sapiens
  Myosin regulatory light chain 2; Accession NO as of 10 Dec 2002 P24844
MSSKRAKAKTTKKRPQRATSNVFAMFDQSQIQEFKEAFNMIDQNRDGFIDKEDLHDMLASLGKNPTDEYLEGMMSEAPGPINFTMFLTMFGEKLNGTDPEDVIRNAFACFDEEASGFIHEDHLRELLTTMGDRFTDEEVDEMYREAPIDKKGNFNYVEFTRILKHGAKDKDD

SEQ ID NO: 49 - protein, 114 aa, Homo sapiens
  Calgranulin B; Accession NO as of 10 Dec 2002 P06702
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 50 - protein, 348 aa, Homo sapiens
  Macrophage capping protein; Accession NO as of 10 Dec 2002 P40121
MYTAIPQSGSPFPGSVQDPGLHVWRVEKLKPVPVAQENQGVFFSGDSYLVLHNGPEEVSHLHLWIGQQSSRDEQGACAVLAVHLNTLLGERPVQHREVQGNESDLFMSYFPRGLKYQEGGVESAFHKTSTGAPAAIKKLYQVKGKKNIRATERALNWDSFNTGDCFILDLGQNIFAWCGGKSNILERNKARDLALAIRDSERQGKAQVEIVTDGEEPAEMIQVLGPKPALKEGNPEEDLTADKANAQAAALYKVSDATGQMNLTKVADSSPFALELLISDDCFVLDNGLCGKIYIWKGRKANEKERQAALQVAEGFISRMQYAPNTQVEILPQGRESPIFKQFFKDWK

SEQ ID NO: 51 - protein, 346 aa, Homo sapiens
  Annexin I; Accession NO as of 10 Dec 2002 P04083
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 52 - protein, 469 aa, Homo sapiens
  Keratin, type II cytoskeletal 7; Accession NO as of 10 Dec 2002 P08729
MSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEQGLRTMQDVVEDFKNKYEDEINRRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRAKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 53 - protein, 836 aa, Homo sapiens
  Osteoblast specific factor 2 precursor; Accession NO as of 10 Dec 2002 Q15063
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 54 - protein, 687 aa, Homo sapiens
  Protein-glutamine gamma-glutamyltransferase; Accession NO P21980
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 55 - protein, 204 aa, Homo sapiens
  Rho GDP-dissociation inhibitor 1; Accession NO as of 10 Dec 2002 P52565
MAEQEPTAEQLAQIAAENEEDEHSVNYKPPAQKSIQEIQELDKDDESLRKYKEALLGRVAVSADPNVPNVVVTGLTLVCSSAPGPLELDLTGDLESFKKQSFVLKEGVEYRIKISFRVNREIVSGMKYIQHTYRKGVKIDKTDYMVGSYGPRAEEYEFLTPVEEAPKGMLARGSYSIKSRFTDDDKTDHLSWEWNLTIKKDWKD

SEQ ID NO: 56 - protein, 492 aa, Homo sapiens
  Fascin 2; Accession NO as of 29 August 2003 O14926
MPTNGLHQVLKIQFGLVNDTDRYLTAESFGFKVNASAPSLKRKQTWVLEPDPGQGTAVLLRSSHLGRYLSAEEDGRVACEAEQPGRDCRFLVLPQPDGRWVLRSEPHGRFFGGTEDQLSCFATAVSPAELWTVHLAIHPQAHLLSVSRRRYVHLCPREDEMAADGDKPWGVDALLTLIFRSRRYCLKSCDSRYLRSDGRLVWEPEPRACYTLEFKAGKLAFKDCDGHYLAPVGPAGTLKAGRNTRPGKDELFDLEESHPQVVLVAANHRYVSVRQGVNVSANQDDELDHETFLMQIDQETKKCTFYSSTGGYWTLVTHGGIHATATQVSANTMFEMEWRGRRVALKASNGRYVCMKKNGQLAAISDFVGKDEEFTLKLINRPILVLRGLDGFVCHHRGSNQLDTNRSVYDVFHLSFSDGAYRIRGRDGGFWYTGSHGSVCSDGERAEDFVFEFRERGRLAIRARSGKYLRGGASGLLRADADAPAGTALWEY

SEQ ID NO: 57 - protein, 165 aa, Homo sapiens
  Destrin (Actin-depolymerizing factor) (ADF); Accession NO as of 29 August 2003 P18282
MASGVQVADEVCRIFYDMKVRKCSTPEEIKKRKKAVIFCLSADKKCIIVEEGKEILVGDVGVTITDPFKHFVGMLPEKDCRYALYDASFETKESRKEELMFFLWAPELAPLKSKMIYASSKDAIKKKFQGIKHECQANGPEDLNRACIAEKLGGSLIVAFEGCPV

SEQ ID NO: 58 - protein, 492 aa, Homo sapiens
  Fascin; Accession NO as of 29 August 2003 Q16658
TANGTAEAVQIQFGLINCGNKYLTAEAFGFKVNASASSLKKKQIWTLEQPPDEAGSAAVCLRSHLGRYLAADKDGNVTCEREVPGPDCRFLIVAHDDGRWSLQSEAHRRYFGGTEDRLSCFAQTVSPAEKWSVHIAMHPQVNIYSVTRKRYAHLSARPADEIAVDRDVPWGVDSLITLAFQDQRYSVQTADHRFLRHDGRLVARPEPATGYTLEFRSGKVAFRDCEGRYLAPSGPSGTLKAGKATKVGKDELFALEQSCAQVVLQAANERNVSTRQGMDLSANQDEETDQETFQLEIDRDTKKCAFRTHTGKYWTLTATGGVQSTASSKNASCYFDIEWRDRRITLRASNGKFVTSKKNGQLAASVETAGDSELFLMKLINRPIIVFRGEHGFIGCRKVTGTLDANRSSYDVFQLEFNDGAYNIKDSTGKYWTVGSDSAVTSSGDTPVDFFFEFCDYNKVAIKVGGRYLKGDHAGVLKASAETVDPASLWEY

SEQ ID NO: 59 - protein, 317 aa, Homo sapiens [sequence omitted]

SEQ ID NO: 60 - protein, 838 aa, Homo sapiens
  hypothetical 88.6 kDa protein; Accession NO as of 29 August 2003 Q96C61
MPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 61 - protein, 433 aa, Homo sapiens
  human alpha enolase; Accession NO as of 29 August 2003 P06733
SILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 62 - protein, 471 aa, Homo sapiens
  Tryptophanyl-tRNA synthetase; Accession NO as of 29 August 2003 P23381
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLQPLIAEHQARRKEVTDEIVKEFMTPRKLSFDFQ

SEQ ID NO: 63 - protein, 106 aa, Homo sapiens
  Ig kappa chain C regionI; Accession NO as of 29 August 2003 P01834
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 758 aa, Homo sapiens
  Mitofilin; Accession NO as of 29 August 2003 Q16891
MLRACQLSGVTAAAQSCLCGKFVLRPLRPCRRYSTSGSSGLTTGKIAGAGLLFVGGGIGGTILYAKWDSHFRESVEKTIPYSDKLFEMVLGPAAYNVPLPKKSIQSGPLKISSVSEVMKESKQPASQLQKQKGDTPASATAPTEAAQIISAAGDTLSVPAPAVQPEESLKTDHPEIGEGKPTPALSEEASSSSIRERPPEEVAARLAQQEKQEQVKIESLAKSLEDALRQTASVTLQAIAAQNAAVQAVNAHSNILKAAMDNSEIAGEKKSAQWRTVEGALKERRKAVDEAADALLKAKEELEKMKSVIENAKKKEVAGAKPHITAAEGKLHNMIVDLDNVVKKVQAAQSEAKVVSQYHELVVQARDDFKRELDSITPEVLPGWKGMSVSDLADKLSTDDLNSLIAHAHRRIDQLNRELAEQKATEKQHITLALEKQKLEEKRAFDSAVAKALEHHRSEIQAEQDRKIEEVRDAMENEMRTQLRRQAAAHTDHLRDVLRVQEQELKSEFEQNLSEKLSEQELQFRRLSQEQVDNFTLDINTAYARLRGIEQAVQSHAVAEEEARKAHQLWLSVEALKYSMKTSSAETPTIPLGSAVEAIKANCSDNEFTQALTAAIPPESLTRGVYSEETLRARFYAVQKLARRVAMIDETRNSLYQYFLSYLQSLLLFPPQQLKPPPELCPEDINTFKLLSYASYCIEHGDLELAAKFVNQLKGESRRVAQDWLKEARMTLETKQIVEILTAYASAVGIGTTQVQPE

SEQ ID NO: 65 - protein, 1410 aa, Homo sapiens
  Ribisome-binding protein 1; Accession NO as of 29 August 2003 O75300
MDIYDTQTLGVVVFGGFMVVSAIGIFLVSTFSMKETSYEEALANQRKEMAKTHHQKVEKKKKEKTVEKKGKTKKKEEKPNGKIPDHDPAPNVTVLLREPVRAPAVAVAPTPVQPPIIVAPVATVPAMPQEKLASSPKDKKKKEKKVAKVEPAVSSVVNSIQVLTSKAAILETAPKEVPMVVVPPVGAKGNTPATGTTQGKKAEGTQNQSKKAEGAPNQGRKAEGTPNQGKKTEGTPNQGKKAEGTPNQGKKAEGTPNQGKKAEGAQNQGKKVDTTPNQGKKVEGAPTQGRKAEGAQNQAKKVEGAQNQGKKAEGAQNQGKKGEGAQNQGKKAEGAQNQGKKAEGAQNQGKKAEGAQNQGKKAEGAQNQGKKAEGAQNQGKKSEGAQNQGKKVEGAQNQGKKAEGAQNQGKKAEGAQNQGKKAEGAQNQGKKAEGAQNQGKKAEGAQNQGKKAEGAQNQGKKAEGAQNQGKKVEGAQNQGKKAEGAQNQGKKAEGAQNQGKKAEGAQNQGQKGEGAQNQGKKTEGAQGKKAERSPNQGKKGEGAPIQGKKADSVANQGTKVEGITNQGKKAEGSPSEGKKAEGSPNQGKKADAAANQGKKTESASVQGRNTDVAQSPEAPKQEAPAKKKSGSKKKGEPGPPDADGPLYLPYKTLVSTVGSMVFNEGEAQRLIEILSEKAGIIQDTWHKATQKGDPVAILKRQLEEKEKLLATEQEDAAVAKSKLRELNKEMAAEKAKAAAGEAKVKKQLVAREQEITAVQARMQASYREHVKEVQQLQGKIRTLQEQLENGPNTQLARLQQENSILRDALNQATSQVESKQNAELAKLRQELSKVSKELVEKSEAVRQDEQQRKALEAKAAAFEKQVLQLQASHRESEEALQKRLDEVSRELCHTQSSHASLRADAEKAQEQQQQMAELHSKLQSSEAEVRSKCEELSGLHGQLQEARAENSQLTERIRSIEALLEAGQARDAQDVQASQAEADQQQTRLKELESQVSGLEKEAIELREAVEQQKVKNNDLREKNWKAMEALATAEQACKEKLLSLTQAKEESEKQLCLIEAQTMEALLALLPELSVLAQQNYTEWLQDLKEKGPTLLKHPPAPAEPSSDLASKLREAEETQSTLQAECDQYRSILAETEGMLRDLQKSVEEEEQVWRAKVGAAEEELQKSRVTVKHLEEIVEKLKGELESSDQVREHTSHLEAELEKHMAAASAECQNYAKEVAGLRQLLLESQSQLDAAKSEAQKQSDELALVRQQLSEMKSHVEDGDIAGAPASSPEAPPAEQDPVQLKTQLEWTEAILEDEQTQRQKLTAEFEEAQTSACRLQEELEKLRTAGPLESSETEEASQLKERLEKEKKLTSDLGRAATRLQELLKTTQEQLAREKDTVKKLQEQLEKAEDGSSSKEGTSV

SEQ ID NO: 66 - protein, 453 aa, Homo sapiens
  Fibrinogen gamma chain precursor; Accession NO as of 29 August 2003 P02679
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 67 - protein, 622 aa, Homo sapiens
  Prothrombin Precursor; Accession NO as of 29 August 2003 P00734
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 68 - protein, 530 aa, Homo sapiens
  Pyruvate kinase; Accession NO as of 29 August 2003 P14618
SKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 69 - protein, 328 aa, Homo sapiens
  Reticulocalbin 3 precursor; Accession NO as of 29 August 2003 Q96D15
MMWRPSVLLLLLLLRHGAQGKPSPDAGPHGQGRVHQAAPLSDAPHDDAHGNFQYDHEAFLGREVAKEFDQLTPEESQARLGRIVDRMDRAGDGDGWVSLAELRAWIAHTQQRHIRDSVSAAWDTYDTDRDGRVGWEELRNATYGHYAPGEEFHDVEDAETYKKMLARDERRFRVADQDGDSMATREELTAFLHPEEFPHMRDIVIAETLEDLDRNKDGYVQVEEYIADLYSAEPGEEEPAWVQTERQQFRDFRDLNKDGHLDGSEVGHWVLPPAQDQPLVEANHLLHESDTDKDGRLSKAEILGNWNMFVGSQATNYGEDLTRHHDEL

SEQ ID NO: 70 - protein, 469 aa, Homo sapiens
  Desmin; Accession NO as of 29 August 2003 P17661
SQAYSSSQRVSSYRRTFGGAPGFPLGSPLSSPVFPRAGFGSKGSSSSVTSRVYQVSRTSGGAGGLGSLRASRLGTTRTPSSYGAGELLDFSLADAVNQEFLTTRTNEKVELQELNDRFANYIEKVRFLEQQNAALAAEVNRLKGREPTRVAELYEEELRELRRQVEVLTNQRARVDVERDNLLDDLQRLKAKLQEEIQLKEEAENNLAAFRADVDAATLARIDLERRIESLNEEIAFLKKVHEEEIRELQAQLQEQQVQVEMDMSKPDLTAALRDIRAQYETIAAKNISEAEEWYKSKVSDLTQAANKNNDALRQAKQEMMEYRHQIQSYTCEIDALKGTNDSLMRQMRELEDRFASEASGYQDNIARLEEEIRHLKDEMARHLREYQDLLNVKMALDVEIATYRKLLEGEESRINLPIQTYSALNFRETSPEQRGSEVHTKKTVMIKTIETRDGEVVSEATQQQHEVL

SEQ ID NO: 71 - protein, 417 aa, Homo sapiens
  Carboxypeptidase B precursor; Accession NO as of 29 August 2003 P15086
MLALLVLVTVALASAHHGGEHFEGEKVFRVNVEDENHINIIRELASTTQIDFWKPDSVTQIKPHSTVDFRVKAEDTVTVENVLKQNELQYKVLISNLRNVVEAQFDSRVRATGHSYEKYNKWETIEAWTQQVATENPALISRSVIGTTFEGRAIYLLKVGKAGQNKPAIFMDCGFHAREWISPAFCQWFVREAVRTYGREIQVTELLDKLDFYVLPVLNIDGYIYTWTKSRFWRKTRSTHTGSSCIGTDPNRNFDAGWCEIGASRNPCDETYCGPAAESEKETKALADFIRNKLSSIKAYLTIHSYSQMMIYPYSYAYKLGENNAELNALAKATVKELASLHGTKYTYGPGATTIYPAAGGSDDWAYDQGIRYSFTFELRDTGRYGFLLPESQIRATCEETFLAIKYVASYVLEHLY

SEQ ID NO: 72 - protein, 419 aa, Homo sapiens
  Carboxypeptidase A1 precursor; Accession NO as of 29 August 2003 P15085
MRGLLVLSVLLGAVFGKEDFVGHQVLRISVADEAQVQKVKELEDLEHLQLDFWRGPAHPGSPIDVRVPFPSIQAVKIFLESHGISYETMIEDVQSLLDEEQEQMFAFRSRARSTDTFNYATYHTLEEIYDFLDLLVAENPHLVSKIQIGNTYEGRPIYVLKFSTGGSKRPAIWIDTGIHSREWVTQASGVWFAKKITQDYGQDAAFTAILDTLDIFLEIVTNPDGFAFTHSTNRMWRKTRSHTAGSLCIGVDPNRNWDAGFGLSGASSNPCSETYHGKFANSEVEVKSIVDFVKDHGNIKAFISIHSYSQLLMYPYGYKTEPVPDQDELDQLSKAAVTALASLYGTKFNYGSIIKAIYQASGSTIDWTYSQGIKYSFTFELRDTGRYGFLLPASQIIPTAKETWLALLTIMEHTLNHPY

SEQ ID NO: 73 - protein, 418 aa, Homo sapiens; identical to SEQ ID NO: 20
  Colligin 2; Accession NO as of 29 August 2003 P50454

SEQ ID NO: 74 - protein, 263 aa, Homo sapiens
  Chymotrypsinogen B precursor; Accession NO as of 29 August 2003 P17538
MAFLWLLSCWALLGTTFGCGVPAIHPVLSGLSRIVNGEDAVPGSWPWQVSLQDKTGFHFCGGSLISEDWVVTAAHCGVRTSDVVVAGEFDQGSDEENIQVLKIAKVFKNPKFSILTVNNDITLLKLATPARFSQTVSAVCLPSADDDFPAGTLCATTGWGKTKYNANKTPDKLQQAALPLLSNAECKKSWGRRITDVMICAGASGVSSCMGDSGGPLVCQKDGAWTLVGIVSWGSDTCSTSSPGVYARVTKLIPWVQKILAAN

SEQ ID NO: 75 - protein, 247 aa, Homo sapiens
  Trypsin I precursor; Accession NO as of 29 August 2003 P07477
MNPLLILTFVAAALAAPFDDDDKIVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 76 - protein, 247 aa, Homo sapiens
  Trypsin II precursor; Accession NO as of 29 August 2003 P07478
MNLLLILTFVAAAVAAPFDDDDKIVGGYICEENSVPYQVSLNSGYHFCGGSLISEQWVVSAGHCYKSRIQVRLGEHNIEVLEGNEQFINAAKIIRHPKYNSRTLDNDILLIKLSSPAVINSRVSAISLPTAPPAAGTESLISGWGNTLSSGADYPDELQCLDAPVLSQAECEASYPGKITNNMFCVGFLEGGKDSCQGDSGGPVVSNGELQGIVSWGYGCAQKNRPGVYTKVYNYVDWIKDTIAANS

SEQ ID NO: 77 - protein, 379 aa, Homo sapiens
  Leukocyte elastase inhibitor (LEI); Accession NO as of 29 August 2003 P30740
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 78 - protein, 573 aa, Homo sapiens
  Hsp60; Accession NO as of 29 August 2003 P10809
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 79 - protein, 803 aa, Homo sapiens
  Endoplasmin precursor (GRP94); Accession NO as of 29 August 2003 P14625
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 80 - protein, 261 aa, Homo sapiens
  Endoplasmic reticulum protein Erp29 precursor (Erp31)(Erp28); Accession NO as of 29 August 2003 P30040
MAAAVPRAAFLSPLLPLLLGFLLLSAPHGGSGLHTKGALPLDTVTFYKVIPKSKFVLVKFDTQYPYGEKQDEFKRLAENSASSDDLLVAEVGISDYGDKLNMELSEKYKLDKESYPVFYLFRDGDFENPVPYTGAVKVGAIQRWLKGQGVYLGMPGCLPVYDALAGEFIRASGVEARQALLKQGQDNLSSVKETQKKWAEQYLKIMGKILDQGEDFPASEMTRIARLIEKNKMSDGKKEELQKSLNILTAFQKKGAEKEEL

SEQ ID NO: 81 - protein, 525 aa, Homo sapiens
  Protein disulfide isomerase A2 precursor; Accession NO as of 29 August 2003 Q13087
MSRQLLPVLLLLLLRASCPWGQEQGARSPSEEPPEEEIPKEDGILVLSRHTLGLALREHPALLVEFYAPWCGHCQALAPEYSKAAAVLAAESMVVTLAKVDGPAQRELAEEFGVTEYPTLKFFRNGNRTHPEEYTGPRDAEGIAEWLRRRVGPSAMRLEDEAAAQALIGGRDLVVIGFFQDLQDEDVATFLALAQDALDMTFGLTDRPRLFQQFGLTKDTVVLFKKFDEGRADFPVDEELGLDLGDLSRFLVTHSMRLVTEFNSQTSAKIFAARILNHLLLFVNQTLAAHRELLAGFGEAAPRFRGQVLFVVVDVAADNEHVLQYFGLKAEAAPTLRLVNLETTKKYAPVDGGPVTAASITAFCHAVLNGQVKPYLLSQEIPPDWDQRPVKTLVGKNFEQVAFDETKNVFVKFYAPWCTHCKEMAPAWEALAEKYQDHEDIIIAELDATANELDAFAVHGFPTLKYFPAGPGRKVIEYKSTRDLETFSKFLDNGGVLPTEEPPEEPAAPFPEPPANSTMGSKEEL

SEQ ID NO: 82 - protein, 505 aa, Homo sapiens
  Protein disulfide isomerase A3 precursor; Accession NO as of 29 August 2003 P30101
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 83 - protein, 374 aa, Homo sapiens
  Alcohol dehydrogenase beta chain; Accession NO as of 29 August 2003 P00325
STAGKVIKCKAAVLWEVKKPFSIEDVEVAPPKAYEVRIKMVAVGICRTDDHVVSGNLVTPLPVILGHEAAGIVESVGEGVTTVKPGDKVIPLFTPQCGKCRVCKNPESNYCLKNDLGNPRGTLQDGTRRFTCRGKPIHHFLGTSTFSQYTVVDENAVAKIDAASPLEKVCLIGCGFSTGYGSAVNVAKVTPGSTCAVFGLGGVGLSAVMGCKAAGAARIIAVDINKDKFAKAKELGATECINPQDYKKPIQEVLKEMTDGGVDFSFEVIGRLDTMMASLLCCHEACGTSVIVGVPPASQNLSINPMLLLTGRTWKGAVYGGFKSKEGIPKLVADFMAKKFSLDALITHVLPFEKINEGFDLLHSGKSIRTVLTF

SEQ ID NO: 84 - protein, 241 aa, Homo sapiens
  Glutathione transferase omega 1; Accession NO as of 29 August 2003 P78417
MSGESARSLGKGSAPPGPVPEGSIRIYSMRFCPFAERTRLVLKAKGIRHEVININLKNKPEWFFKKNPFGLVPVLENSQGQLIYESAITCEYLDEAYPGKKLLPDDPYEKACQKMILELFSKVPSLVGSFIRSQNKEDYAGLKEEFRKEFTKLEEVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 85 - protein, 999 aa, Homo sapiens
  150 kDa oxygen-regulated protein precursor (Orp150); Accession NO as of 29 August 2003 Q9Y4L1
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 86 - protein, 271 aa, Homo sapiens
  Peroxiredoxin 4; Accession NO as of 29 August 2003 Q13162
MEALPLLAATTPDHGRHRRLLLLPLLLFLLPAGAVQGWETEERPRTREEECHFYAGGQVYPGEASRVSVADHSLHLSKAKISKPAPYWEGTAVIDGEFKELKLTDYRGKYLVFFFYPLDFTFVCPTEIIAFGDRLEEFRSINTEVVACSVDSQFTHLAWINTPRRQGGLGPIRIPLLSDLTHQISKDYGVYLEDSGHTLRGLFIIDDKGILRQITLNDLPVGRSVDETLRLVQAFQYTDKHGEVCPAGWKPGSETIIPDPAGKLKYFDKLN

SEQ ID NO: 87 - protein, 288 aa, Homo sapiens
  MAWD binding protein; Accession NO as of 29 August 2003 P30039
MKLPIFIADAFTARAFRGNPAAVCLLENELDEDMHQKIAREMNLSETAFIRKLHPTDNFAQSSCFGLRWFTPASEVPLCGHATLASAAVLFHKIKNMNSTLTFVTLSGELRARRAEDGIVLDLPLYPAHPQDFHEVEDLIKTAIGNTLVQDICYSPDTQKLLVRLSDVYNRSFLENLKVNTENLLQVENTGKVKGLILTLKGEPGGQTQAFDFYSRYFAPWVGVAEDPVTGSAHAVLSSYWSQHLGKKEMHAFQCSHRGGELGISLRPDGRVDIRGGAAVVLEGTLTA

SEQ ID NO: 88 - protein, 511 aa, Homo sapiens
  Alpha-amylase 2B precursor; Accession NO as of 29 August 2003 P19961
MKFFLLLFTIGFCWAQYSPNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMSGNAVSAGTSSTCGSYFNPGSRDFPAVPYSGWDFNDGKCKTGSGDIENYNDATQVRDCRLVGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 89 - protein, 511 aa, Homo sapiens
  Alpha-amylase, pancreatic precursor; Accession NO as of 29 August 2003 P04746
MKFFLLLFTIGFCWAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 90 - protein, 553 aa, Homo sapiens
  ATP synthase alpha chain; Accession NO as of 29 August 2003 P25705
MLSVRVAAAVVRALPRRAGLVSRNALGSSFIAARNFHASNTHLQKTGTAEMSSILEERILGADTSVDLEETGRVLSIGDGIARVHGLRNVQAEEMVEFSSGLKGMSLNLEPDNVGVVVFGNDKLIKEGDIVKRTGAIVDVPVGEELLGRVVDALGNAIDGKGPIGSKTRRRVGLKAPGIIPRISVREPMQTGIKAVDSLVPIGRGQRELIIGDRQTGKTSIAIDTIINQKRFNDGSDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDAFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVVSQHQALLGTIRADGKISEQSDAKLKEIVTNFLAGFEA

SEQ ID NO: 91 - protein, 742 aa, Homo sapiens
  Bile-salt-activated lipase precursor; Accession NO as of 29 August 2003 P19835
MGRLQLVVLGLTCCWAVASAAKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPTVTDQEATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPVPPTGDSGAPPVPPTGDSGAPPVTPTGDSETAPVPPTGDSGAPPVPPTGDSEAAPVPPTDDSKEAQMPAVIRF

SEQ ID NO: 92 - protein, 467 aa, Homo sapiens
  Pancreatic lipase related protein precursor; Accession NO as of 29 August 2003 P54315
MLIFWTITLFLLGAAKGKEVCYEDLGCFSDTEPWGGTAIRPLKILPWSPEKIGTRFLLYTNENPNNFQILLLSDPSTIEASNFQMDRKTRFIIHGFIDKGDESWVTDMCKKLFEVEEVNCICVDWKKGSQATYTQAANNVRVVGAQVAQMLDILLTEYSYPPSKVHLIGHSLGAHVAGEAGSKTPGLSRITGLDPVEASFESTPEEVRLDPSDADFVDVIHTDAAPLIPFLGFGTNQQMGHLDFFPNGGESMPGCKKNALSQIVDLDGIWAGTRDFVACNHLRSYKYYLESILNPDGFAAYPCTSYKSFESDKCFPCPDQGCPQMGHYADKFAGRTSEEQQKFFLNTGEASNFARWRYGVSITLSGRTATGQIKVALFGNKGNTHQYSIFRGILKPGSTHSYEFDAKLDVGTIEKVKFLWNNNVINPTLPKVGATKITVQKGEEKTVYNFCSEDTVREDTLLTLTPC

SEQ ID NO: 93 - protein, 469 aa, Homo sapiens
  Pancreatic lipase related protein 2 precursor; Accession NO as of 29 August 2003 P54317
MLPPWTLGLLLLATVRGKEVCYGQLGCFSDEKPWAGTLQRPVKLLPWSPEDIDTRFLLYTNENPNNFQLITGTEPDTIEASNFQLDRKTRFIIHGFLDKAEDSWPSDMCKKMFEVEKVNCICVDWRHGSRAMYTQAVQNIRVVGAETAFLIQALSTQLGYSLEDVHVIGHSLGAHTAAEAGRRLGGRVGRITGLDPAGPCFQDEPEEVRLDPSDAVFVDVIHTDSSPIVPSLGFGMSQKVGHLDFFPNGGKEMPGCKKNVLSTITDIDGIWEGIGGFVSCNHLRSFEYYSSSVLNPDGFLGYPCASYDEFQESKCFPCPAEGCPKMGHYADQFKGKTSAVEQTFFLNTGESGNFTSWRYKVSVTLSGKEKVNGYIRIALYGSNENSKQYEIFKGSLKPDASHTCAIDVDFNVGKIQKVKFLWNKRGINLSEPKLGASQITVQSGEDGTEYNFCSSDTVEENVLQSLYPC

SEQ ID NO: 94 - protein, 465 aa, Homo sapiens
  Triacylglycerol lipase, pancreatic precursor; Accession NO as of 29 August 2003 P16233
MLPLWTLSLLLGAVAGKEVCYERLGCFSDDSPWSGITERPLHILPWSPKDVNTRFLLYTNENPNNFQEVAADSSSISGSNFKTNRKTRFIIHGFIDKGEENWLANVCKNLFKVESVNCICVDWKGGSRTGYTQASQNIRIVGAEVAYFVEFLQSAFGYSPSNVHVIGHSLGAHAAGEAGRRTNGTIGRITGLDPAEPCFQGTPELVRLDPSDAKFVDVIHTDGAPIVPNLGFGMSQVVGHLDFFPNGGVEMPGCKKNILSQIVDIDGIWEGTRDFAACNHLRSYKYYTDSIVNPDGFAGFPCASYNVFTANKCFPCPSGGCPQMGHYADRYPGKTNDVGQKFYLDTGDASNFARWRYKVSVTLSGKKVTGHILVSLFGNKGNSKQYEIFKGTLKPDSTHSNEFDSDVDVGDLQMVKFIWYNNVINPTLPRVGASKIIVETNVGKQFNFCSPETVREEVLLTLTPC

SEQ ID NO: 95 - protein, 572 aa, Homo sapiens
  Dihydropyrimidinase related protein-2; Accession NO as of 29 August 2003 Q16555
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 96 - protein, 500 aa, Homo sapiens
  4-aminobutyrate aminotransferase, mitochondrial Precursor; Accession NO as of 29 August 2003 P80404
MASMLLAQRLACSFQHTYRLLVPGSRHISQAAAKVDVEFDYDGPLMKTEVPGPRSQELMKQLNIIQNAEAVHFFCNYEESRGNYLVDVDGNRMLDLYSQISSVPIGYSDPALVKLIQQPQNASMFVNRPALEILPPENFVEKLRQSLLSVAPKGMSQLITMACGSCSNENALKTIFMWYRSKERGQRGFSKEELETCMINQAPWCPDYSILSFMGSFHGRTMGCLATTHSKAIHKIDIPSFDWPIAPFPRLKYPLEEFVKENQQEEAGCLEEVEDLIVKYRKKKKTVAGIIVEPIQSEGGDNHASDDFFRKLRDIARKHCCAFLVDEVQTGGGCTGKFWAHEHWGLDDPADVMTFSKKMMTGGFFLKEEFRPNAPYRIFNTWLGDPSKNLLLAEVINIIKREDLLNNAAHAGKALLTGLLDLQARYPQFISRVRGRGTFCSFDTPDDSIRNKLILIARNKGVVLGGCGDKSIRFRPTLVFRDHHAHLFLNIFSDILADFK

SEQ ID NO: 97 - protein, 423 aa, Homo sapiens
  Glycine amidiontransferase, mitochondrial Precursor; Accession NO as of 29 August 2003 P50440
MLRVRCLRGGSRGAEAVHYIGSRLGRTLTGWVQRTFQSTQAATASSRNSCAADDKATEPLPKDCPVSSYNEWDPLEEVIVGRAENACVPPFTIEVKANTYEKYWPFYQKQGGHYFPKDHLKKAVAEIEEMCNILKTEGVTVRRPDPIDWSLKYKTPDFESTGLYSAMPRDILIVVGNEIIEAPMAWRSRFFEYRAYRSIIKDYFHRGAKWTTAPKPTMADELYNQDYPIHSVEDRHKLAAQGKFVTTEFEPCFDAADFIRAGRDIFAQRSQVTNYLGIEWMRRHLAPDYRVHIISFKDPNPMHIDATFNIIGPGIVLSNPDRPCHQIDLFKKAGWTIITPPTPIIPDDHPLWMSSKWLSMNVLMLDEKRVMVDANEVPIQKMFEKLGITTIKVNIRNANSLGGGFHCWTCDVRRRGTLQSYLD

SEQ ID NO: 98 - protein, 654 aa, Homo sapiens
  GRP 78; Accession NO as of 29 August 2003 P11021
MKLSLVAAMLLLLLSAARAKEEDMGTVVAIHLGTTYPCVGVFKNGRMEIIANDQGNRIMPSYVAFTPEGECLIGDAAKNQLTSNPKNTVFDAKRLIGRRWHDPSVQQDIEFLPFKVVEKNTKSYIQIDVGGGQTKTFAPKEISAMVLTKMKENAEAYLGKVTHAVVTAPAYFNDAQCQATKDAGTIADLNVMRIINKPTAAAIAYGLDKREGEKNILVFDLGGGTFDVSLLTIDNGVFKVVATNGDTYLGGEDFDQRVMEHFIKLYKKKTGKDVRKDNRAVQKLWRKVEKAKRALSSQHQAXVIEIESFYEGEDFSETLTQAKFEELNXDLFQSTMKPSQRSVXKVLEDSDLKKSDIDETVLVGGFTQIPKIQQLVKEFFNGKELSRGISPYEAVAYGAAVQAGVLSGDQDTGDLVLLDICPLTLGIETVGGVMTKLIPRNTVVPTKKSQIFSTAFDNQPXTIKVYEGKQPLTKDNHLLGTFDLTGIPPAPCGVPQIEVTFEMDVSDILQVTAKDKGTRYKNKITITNDQNHLTPEDIERMVNDAEKFAEEDKKLKECTDTRNELESYAYSLKNQIGDKEKLGGKLSSEDKETMEKTVEEKTEWLESHQDADTEDFKAKKKELEEIVQPIISKLYGSAGPPPTGEEDTAEKDEL

SEQ ID NO: 99 - protein, 325 aa, Homo sapiens
  Eukaryotic translation initiation factor 3 subunit 2; Accession NO as of 29 August 2003 Q13347
MKPILLQGHERSITQIKYNREGDLLFTVAKDPIVNVWYSVNGERLGTYMGHTGAVWCVDADWDTKHVLTGSADNSCRLWDCETGKQLALLKTNSAVRTCGFDFGGNIIMFSTDKQMGYQCFVSFFDLRDPSQIDNNEPYMKIPCNDSKITSAVWGPLGECIIAGHESGELNQYSAKSGEVLVNVKEHSRQINDIQLSRDMTMFVTASKDNTAKLFDSTTLEHQKTFRTERPVNSAALSPNYDHVVLGGGQEAMDVTTTSTRIGKFEARFFHLAFEEEFGRVKGHFGPINSVAFHPDGKSYSSGGEDGYVRIHYFDPQYFEFEFEA

SEQ ID NO: 100 - protein, 572 aa, Homo sapiens; identical to SEQ ID NO: 95
  Dihydropyrimidinase related protein-2; Accession NO as of 29 August 2003 Q16555

SEQ ID NO: 101 - protein, 561 aa, Homo sapiens
  Phosphoglucomutase (EC 5.4.2.2) (Glucose phosphomutase) (PGM); Accession NO as of 29 August 2003 P36871
VKIVTVKTQAYQDQKPGTSGLRKRVKVFQSSANYAENFIQSIISTVEPAQRQEATLVVGGDGRFYMKEAIQLIARIAAANGIGRLVIGQNGILSTPAVSCIIRKIKAIGGIILTASHNPGGPNGDFGIKFNISNGGPAPEAITDKIFQISKTIEEYAVCPDLKVDLGVLGKQQFDLENKFKPFTVEIVDSVEAYATMLRSIFDFSALKELLSGPNRLKIRIDAMHGVVGPYVKKILCEELGAPANSAVNCVPLEDFGGHHPDPNLTYAADLVETMKSGEHDFGAAFDGDGDRNMILGKHGFFVNPSDSVAVIAANIFSIPYFQQTGVRGFARSMPTSGALDRVASATKIALYETPTGWKFFGNLMDASKLSLCGEESFGTGSDHIREKDGLWAVLAWLSILATRKQSVEDILKDHWQKYGRNFFTRYDYEEVEAEGANKMMKDLEALMFDRSFVGKQFSANDKVYTVEKADNFEYSDPVDGSISRNQGLRLIFTDGSRIVFRLSGTGSAGATIRLYIDSYEKDVAKINQDPQVMLAPLISIALKVSQLQERTGRTAPTVIT

SEQ ID NO: 102 - protein, 263 aa, Homo sapiens
  Proteasome subunit alpha type 1; Accession NO as of 29 August 2003 P25786
MFRNQYDNDVTVWSPQGRIHQIEYAMEAVKQGSATVGLKSKTHAVLVALKRAQSELAAHQKKILHVDNHIGISIAGLTADARLLCNFMRQECLDSRFVFDRPLPVSRLVSLIGSKTQIPTQRYGRRPYGVGLLIAGYDDMGPHIFQTCPSANYFDCRAMSIGARSQSARTYLERHMSEFMECNLNELVKHGLRALRETLPAEQDLTTKNVSIGIVGKDLEFTIYDDDDVSPFLEGLEERPQRKAQPAQPADEPAEKADEPMEH

SEQ ID NO: 103 - protein, 205 aa, Homo sapiens
  Heat shock 27 kDa protein; Accession NO as of 29 August 2003 P04792
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 104 - protein, 868 aa, Homo sapiens
  Programmed cell death 6 interacting protein (Hp95); Accession NO as of 29 August 2003 Q8WUM4
MATFISVQLKKTSEVDLAKPLVKFIQQTYPSGGEEQAQYCRAAEELSKLRRAAVGRPLDKHEGALETLLRYYDQICSIEPKFPFSENQICLTFTWKDAFDKGSLFGGSVKLALASLGYEKSCVLFNCAALASQIAAEQNLDNDEGLKIAAKHYQFASGAFLHIKETVLSALSREPTVDISPDTVGTLSLIMLAQAQEVFFLKATRDKMKDAIIAKLANQAADYFGDAFKQCQYKDTLPKEVFPVLAAKHCIMQANAEYHQSILAKQQKKFGEEIARLQHAAELIKTVASRYDEYVNVKDFSDKINRALAAAKKDNDFIYHDRVPDLKDLDPIGKATLVKSTPVNVPISQKFTDLFEKMVPVSVQQSLAAYNQRKADLVNRSIAQMREATTLANGVLASLNLPAAIEDVSGDTVPQSILTKSRSVIEQGGIQTVDQLIKELPELLQRNREILDESLRLLDEEEATDNDLRAKFKERWQRTPSNELYKPLRAEGTNFRTVLDKAVQADGQVKECYQSHRDTIVLLCKPEPELNAAIPSANPAKTMQGSEVVNVLKSLLSNLDEVKKEREGLENDLKSVNFDMTSKFLTALAQDGVINEEALSVTELDRVYGGLTTKVQESLKKQEGLLKNIQVSHQEFSKMKQSNNEANLREEVLKNLATAYDNFVELVANLKEGTKFYNELTEILVRFQNKCSDIVFARKTERDELLKDLQQSIAREPSAPSIPTPAYQSSPAGGHAPTPPTPAPRTMPPTKPQPPARPPPPVLPANRAPSATAPSPVGAGTAAPAPSQTPGSAPPPQAQGPPYPTYPGYPGYCQMPMPMGYNPYAYGQYNMPYPPVYHQSPGQAPYPGPQQPSYPFPQPPQQSYYPQQ

SEQ ID NO: 105 - protein, 280 aa, Homo sapiens
  Similar to four and a half LIM domains 3; Accession NO as of 29 August 2003 Q9BVA2
MSESFDCAKCNESLYGRKYIQTDSGPYCVPCYDNTFANTCAECQQLIGHDSRELFYEDRHFHEGCFRCCRCQRSLADEPFTCQDSELLCNDCYCSAFSSQCSACGETVMPGSRKLEYGGQTWHEHCFLCSGCEQPLGSRSFVPDKGAHYCVPCYENKFAPRCARCSKTLTQGGVTYRDQPWHRECLVCTGCQTPLAGQQFTSRDEDPYCVACFGELFAPKCSSCKRPIVGLGGGKYVSFEDRHWHHNCFSCARCSTSLVGQGFVPDGDQVLCQGCSQAGP

SEQ ID NO: 106 - protein, 280 aa, Homo sapiens
  Skeletal muscle LIM-protein 2 (SLIM 2); Accession NO as of 29 August 2003 Q13643
MSESFDCAKCNESLYGRKYIQTDSGPYCVPCYDNTFANTCAECQQLIGHDSRELFYEDRHFHEGCFRCCRCQRSLADEPFTRQDSELLCNDCYCSAFSSQCSACGETVMPGSRKLEYGGQTWHEHCFLCIGCEQPLGSRPFVPDKGAHYCVPCYENNFAPRCARCTKTLTQGGLTYRDLPWHPKCLVCTGCQTPLAGQQFTSRDEDPYCVACFGELFAPKCSSCKRPIVGLGGGKYVSFEDRHWHHNCFTCDRCSNSLVGQGFVPDGDQVLCQGCSQAGP

SEQ ID NO: 107 - protein, 133 aa, Homo sapiens
  Cytochrome b5; Accession NO as of 29 August 2003 P00167
AEQSDEAVKYYTLEEIQKHNHSKSTWLILHHKVYDLTKFLEEHPGGEEVLREQAGGDATENFEDVGHSTDAREMSKTFIIGELHPDDRPKLNKPPETLITTIDSSSSWWTNWVIPAISAVAVALMYRLYMAED

SEQ ID NO: 108 - protein, 175 aa, Homo sapiens
  Pancreatitis-associated protein 1 precursor; Accession NO as of 29 August 2003 Q06141
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 109 - protein, 1028 aa, Homo sapiens
  Collagen alpha 1(VI) chain precursor; Accession NO as of 29 August 2003 P12109
MRAARALLPLLLQACWTAAQDEPETPRAVAFQDCPVDLFFVLDTSESVALRLKPYGALVDKVKSFTKRFIDNLRDRYYRCDRNLVWNAGALHYSDEVEIIQGLTRMPGGRDALKSSVDAVKYFGKGTYTDCAIKKGLEQLLVGGSHLKENKYLIVVTDGHPLEGYKEPCGGLEDAVNEAKHLGVKVFSVAITPDHLEPRLSIIATDHTYRRNFTAADWGQSRDAEEAISQTIDTIVDMIKNNVEQVCCSFECQPARGPPGLRGDPGFEGERGKPGLPGEKGEAGDPGRPGDLGPVGYQGMKGEKGSRGEKGSRGPKGYKGEKGKRGIDGVDGVKGEMGYPGLPGCKGSPGFDGIQGPPGPKGDPGAFGLKGEKGEPGADGEAGRPGARGPSGDEGPAGEPGPPGEKGEAGDEGNPGPDGAPGERGGPGERGPRGTPGPRGPRGDPGEAGPQGDQGREGPVGVPGDPGEAGPIGPKGYRGDEGPPGSEGARGAPGPAGPPGDPGLMGERGEDGPAGNGTEGFPGFPGYPGNRGAPGINGTKGYPGLKGDEGEAGDPGDDNNDIAPRGVKGAKGYRGPEGPQGPPGHQGPPGPDECEILDIIMKMCSCCECKCGPIDLLFVLDSSESIGLQNFEIAKDFVVKVIDRLSRDELVKFEPGQSYAGVVQYSHSQMQEHVSLRSPSIRNVQELKEAIKSLQWMAGGTFTGEALQYTRDQLLPPSPNNRIALVITDGRSDTQRDTTPLNVLCSPGIQVVSVGIKDVFDFIPGSDQLNVISCQGLAPSQGRPGLSLVKENYAELLEDAFLKNVTAQICIDKKCPDYTCPITFSSPADITILLDGSASVGSHNFDTTKRFAKRLAERFLTAGRTDPAHDVRVAVVQYSGTGQQRPERASLQFLQNYTALASAVDAMDFINDATDVNDALGYVTRFYREASSGAAKKRLLLFSDGNSQGATPAAIEKAVQEAQRAGIEIFVVVVGRQVNEPHIRVLVTGKTAEYDVPYGESHLFRVPSYQALLRGVFHQTVSRKVALG

SEQ ID NO: 110 - protein, 338 aa, Homo sapiens
  Lumican precursor; Accession NO as of 29 August 2003 P51884
MSLSAFTLFLALIGGTSGQYYDYDFPLSIYGQSSPNCAPECNCPESYPSAMYCDELKLKSVPMVPPGIKYLYLRNNQIDHIDEKAFENVTDLQWLILDHNLLENSKIKGRVFSKLKQLKKLHINHNNLTESVGPLPKSLEDLQLTHNKITKLGSFEGLVNLTFIHLQHNRLKEDAVSAAFKGLKSLEYLDLSFNQIARLPSGLPVSLLTLYLDNNKISNIPDEYFKRFNALQYLRLSHNELADSGIPGNSFNVSSLVELDLSYNKLKNIPTVNENLENYYLEVNQLEKFDIKSFCKILGPLSYSKIKHLRLDGNRISETSLPPDMYECLRVANEVTLN

(19) United States
(12) Patent Application Publication
(10) Pub. No.: US 2004/0219572 A1
(43) Pub. Date: Nov. 4, 2004

(54) Specific markers for pancreatic cancer

(76) Inventors: Jie Chen, Beijing (CN); Liping Hu, Beijing (CN); Tong Hua Liu, Beijing (CN); Zhao Hui Lu, Beijing (CN); Yan Shen, Beijing (CN)

Correspondence Address: HOFFMANN-LA ROCHE INC., PATENT LAW DEPARTMENT, 340 KINGSLAND STREET, NUTLEY, NJ, 07110

(21) Appl. No.: 10/733,969

(22) Filed: Dec. 11, 2003

(30) Foreign Application Priority Data
Dec. 17, 2002 (EP) 02028058.2
Nov. 5, 2003 (EP) 03025237.3

(51) Int. Cl. C12Q 1/68; G01N 33/574

(52) U.S. Cl. 435/6; 435/7.23

(57) ABSTRACT

The present invention provides polypeptides which are up- or down-regulated in pancreatic cancer and which can be used as markers for diagnosis of pancreatic cancer. The invention also provides an in vitro method for the diagnosis of pancreatic cancer and/or the susceptibility to pancreatic cancer comprising the steps of a) obtaining a biological sample; and b) detecting and/or measuring the increase of one or more polypeptides as disclosed herein. Furthermore, screening methods relating to inhibitors and antagonists of the specific polypeptides disclosed herein are provided.

### BACKGROUND OF THE INVENTION

The present invention relates to markers for diagnosis of pancreatic cancer comprising at least one polypeptide identified by proteomics to be up-regulated in pancreatic cancer, to an in vitro method for the diagnosis of pancreatic cancer and/or the susceptibility to pancreatic cancer comprising the steps of a) obtaining a biological sample; and b) detecting and/or measuring the increase of specific markers as disclosed herein. Furthermore, screening methods relating to antagonists of the specific markers disclosed herein are provided.

Pancreatic cancer is a common cause of death in the Western world. It is one of the most aggressive malignant tumors, with an overall 5-year survival rate of 0.4%. In many patients with pancreatic cancer, accurate preoperative diagnosis is difficult to achieve with conventional imaging analyses. Most patients with pancreatic cancer present late in the course of the disease and have either locally extensive or metastatic disease. Overall, only up to 20% are candidates for resection and have the potential for curative surgery. Among the causes for this late presentation is the lack of diagnostic methods for an earlier detection of the disease. Besides this lack of diagnostic methods, the high mortality of patients with pancreatic cancer is additionally caused by a lack of effective treatments. Therefore, the identification of new targets for early diagnosis of pancreatic tumors, and for the development of agents to treat pancreatic cancer is a challenge of paramount importance.

### SUMMARY OF THE INVENTION

The problem of identifying polypeptides suitable as markers of pancreatic cancer for early diagnosis of the disease, and the long felt need for such markers, was overcome by the present invention by applying the new technology of proteomics. It was surprisingly found by using proteomic technology that a specific set of polypeptides are differentially expressed in pancreatic tissue obtained from individuals suffering from pancreatic cancer, as compared to healthy pancreatic tissue. The differentially expressed polypeptides are listed in appended tables 2 and 3. The polypeptides in table 3 are encoded by genes which were previously identified to be up-regulated in pancreatic cancer on the transcriptional level (Iacobuzio-Donahue et al., (2002), Am. J. Pathol. 160, 1239-1249). However, it is well known that regulation on the transcriptional level is not necessarily indicative of a similar regulation of the expression of the respective gene on the translational level. Thus, only by demonstrating that the polypeptides listed in table 3 are up-regulated in pancreatic cancer is it possible to use them for polypeptide-based diagnostic assays for the detection of pancreatic cancer.

According to one embodiment of the present invention, there is provided a marker for diagnosis of pancreatic cancer comprising at least one polypeptide selected from the group consisting of the polypeptides listed in tables 2, 3 and 6.

According to another embodiment of the present invention, there is provided an in vitro method for the diagnosis of pancreatic cancer and/or the susceptibility to pancreatic cancer comprising the steps of a) obtaining a biological sample; and b) detecting and/or measuring the increase of at least one marker selected from the polypeptides listed in tables 2, 3 and 6.

According to a further embodiment of the present invention, there is provided an in vitro method for the diagnosis of pancreatic cancer and/or the susceptibility to pancreatic cancer comprising the steps of a) obtaining a biological sample; and b) detecting and/or measuring the increase of at least one nucleic acid coding for at least one marker selected from the polypeptides listed in tables 2, 3 and 6.

According to still another embodiment of the present invention, there is provided a screening method for identifying and/or obtaining a compound which interacts with a polypeptide listed in tables 2, 3 and/or 6 whose expression is upregulated in pancreatic cancer, comprising the steps of a) contacting the polypeptide with a compound or a plurality of compounds under conditions which allow interaction of the compound with the polypeptide; and b) detecting the interaction between the compound or plurality of compounds with the polypeptide.

According to yet another embodiment of the present invention, there is provided a screening method for identifying and/or obtaining a compound which is an inhibitor of the expression of a polypeptide listed in tables 2, 3 and/or 6 whose expression is upregulated in pancreatic cancer, comprising the steps of a) contacting a host which expresses the polypeptide with a compound; b) determining the expression level and/or activity of the polypeptide; c) determining the expression level and/or activity of the polypeptide in the host as defined in (a), which has not been contacted with the compound; and d) quantitatively relating the expression level of the polypeptide as determined in (b) and (c), wherein a decreased expression level determined in (b) in comparison to (c) is indicative for an inhibitor of the expression of the polypeptide.

According to yet a further embodiment of the present invention, there is provided a kit for the diagnosis of pancreatic cancer containing one of a) a nucleic acid coding for at least one marker from tables 2, 3 and 6; b) an antibody, or antigen-binding fragment thereof, which binds at least one of the polypeptides listed in tables 2 and 3; or c) a compound that activates or inhibits at least one of the polypeptides listed in tables 2 and 3.

These and other features, aspects and advantages of the present invention will become better understood with reference to the following description and claims.

### DETAILED DESCRIPTION OF THE INVENTION

Based on the polypeptides listed in tables 2 and 3, one embodiment of the present invention provides a marker for diagnosis of pancreatic cancer comprising at least one polypeptide selected from the group consisting of the polypeptides listed in tables 2 and/or 3 (SEQ ID NO: 1 to 24 and 26 to 49; and/or SEQ ID NO: 25 and 50 to 55).

The term “marker” as used herein refers to one or more polypeptides that are regulated in cancer and that can be used to diagnose pancreatic cancer or a susceptibility to pancreatic cancer either alone or as combinations of multiple polypeptides that are known to be regulated in pancreatic cancer. Preferably, the polypeptides are selected from the group consisting of SEQ. ID NO: 2 to 10, 12 to 15, 17, 19, 20, 23, 24, 27, 28, 31 to 40, 42 to 45, 47 and 48; and/or SEQ ID NO: 25 and 50 to 54. More preferably, the polypeptides are selected from the group consisting of SEQ ID NO: 3, 4, 6, 9, 14, 15, 27, 31 to 35, 37, 39, 40; and/or SEQ ID NO: 50 to 52. Even more preferably, the polypeptides are selected from the group consisting of SEQ ID NO: 4, 6, 9, 14, 15, 31, 33 to 35 and/or SEQ ID NO: 51 and 52. Most preferably, the polypeptides are selected from the group consisting of SEQ ID NO: 4, 6, 14, 15 and 31; and/or SEQ ID NO: 52.

The term “polypeptide” as used herein, refers to a polymer of amino acids, and not to a specific length. Thus, peptides, oligopeptides and proteins are included within the definition of polypeptide.

Preferably, the marker of this invention is a marker comprising at least one polypeptide selected from the group consisting of the polypeptides listed in table 2.

Furthermore, a polypeptide selected from the group consisting of the polypeptides listed in tables 2 and/or 3, may be used as a marker or as part of a marker for diagnosis of pancreatic cancer and/or the susceptibility to pancreatic cancer. Preferably, the polypeptides are selected from the group consisting of SEQ. ID NO: 2 to 10, 12 to 15, 17, 19, 20, 23, 24, 27, 28, 31 to 40, 42 to 45, 47 and 48 from table 2 and/or SEQ ID NO: 25 and 50 to 54 from table 3. These polypeptides are induced at least two fold, as can be seen in tables 2 and 3. More preferably, the polypeptides are selected from the group consisting of SEQ ID NO: 3, 4, 6, 9, 14, 15, 27, 31 to 35, 37, 39, 40 from table 2 and/or SEQ ID NO: 50 to 52 from table 3. These polypeptides are induced at least three fold, as can be seen in tables 2 and 3. Even more preferably, the polypeptides are selected from the group consisting of SEQ ID NO: 4, 6, 9, 14, 15, 31, 33 to 35 from table 2 and/or SEQ ID NO: 51 and 52 from table 3. These polypeptides are induced at least 4 fold, as can be seen in tables 2 and 3. Most preferably, the polypeptides are selected from the group consisting of SEQ ID NO: 4, 6, 14, 15 and 31 from table 2 and/or SEQ ID NO: 52 from table 3, which are the polypeptides that are induced five fold, as shown in tables 2 and 3.

According to another embodiment, the present invention pertains to a marker for diagnosis of pancreatic cancer comprising at least one polypeptide selected from the group consisting of the polypeptides listed in table 6. Preferably, the at least one polypeptide additionally does not include SEQ ID NO:s 25 and 50 to 55.

In a preferred embodiment, the marker hereinbefore described additionally comprises at least one of the polypeptides listed in table 5.

Several groups of polypeptides were identified as markers for pancreatic cancers:

Enzymes

One of the enzymes that caught our attention was Glutamine γ-glutamyltransferase/tissue transglutaminase (TGLC, SEQ ID NO: 54). It is a member of the transglutaminase family that catalyzes Ca2+ dependent reactions resulting in the post translational modification (cross-linking and conjugation with polyamines) of proteins at the level of glutamine and lysine residues (Greenberg, C. S., Birckbichler, P. J., and Rice, R. H. Transglutaminases: multifunctional cross-linking enzymes that stabilize tissues. FASEB J., 5: 3071-3077, 1991). Many different roles for this protein have been described, among them apoptosis, adhesion, and differentiation (Amendola, A., Fesus, L., Piacentini, M., and Szondy, Z. “Tissue” transglutaminase in AIDS. J. Immunol. Methods, 265: 145-159, 2002). There is some controversy on the role of TGLC in apoptosis. While several pieces of evidence suggest that TGLC is a pro-apoptotic protein (Melino, G., et al., Mol. Cell Biol., 14: 6584-6596, 1994), Jason et al. found that TGLC acts in anti-apoptotic fashion (Boehm, J. E., et al. J. Biol. Chem., 277: 20127-20130, 2002). Many substrates of TGLC are major extra-cellular matrix (ECM) components such as fibronectin, osteonectin, and collagen, which makes TGLC an important enzyme in ECM development (Raghunath, M., et al., J. Clin. Invest, 98: 1174-1184, 1996., Nemes, Z., Jr., et al. J. Biol. Chem., 272: 20577-20583, 1997). Abnormal ECM development is involved in many pathological conditions such as fibrosis and may play a role in the proliferation of fibrous tissue observed in PC. Haroon et al. described that TGLC ECM-promoting abilities are an important part of the host response mechanism against tumor growth (Haroon, Z. A., et al., Lab Invest, 79: 1679-1686, 1999). Interestingly, loss of TGLC can be a biomarker for prostate adenocarcinoma (Birckbichler, P. J., et al., Cancer, 89: 412-423, 2000), which raises the question whether the measured TGLC is produced by neoplastic ductal cells and/or stromal cells. Measurements of mRNA levels in PC, normal tissue and PC cell lines indicate that TGLC is over expressed in both cell types (Iacobuzio-Donahue, C. A., et al., Am. J. Pathol., 160: 1239-1249, 2002), which would distinguish PC from prostate adenocarcinoma. Therefore, one preferred embodiment of the present invention is a marker comprising SEQ ID NO: 54.

Cytoskeletal Proteins

Several cytoskeletal proteins were detected at higher levels in PC than in surrounding tissue. One of these is gelsolin (SEQ ID NO: 3), a Ca²⁺ and PIP2 (polyphosphoinositide 4,5-bisphosphate) regulated severing and capping protein, which is a multifunctional actin regulatory protein and has roles in actin remodeling, motility, signaling, apoptosis and cancer (Maruta, H. G proteins cytoskeleton and cancer. Austin, Tex.: R. G. Landes, 1998). In several cancer studies, gelsolin expression has been described as down-regulated during carcinogenesis (breast, colon, stomach, bladder, prostate, and lung) (Asch, H. L., et al., Cancer Res., 56: 4841-4845, 1996; Dosaka-Akita, H., et al., Cancer Res., 58: 322-327, 1998, Prasad, S. C., et al. Electrophoresis, 18: 629-637, 1997). Another example for an up-regulated cytoskeletal protein is fascin (SEQ ID NO: 58), an actin-bundling protein that has a role in cell matrix adhesion, cell interaction and migration. Fascin over expression has been reported in several cancers, such as breast, colon, and ovarian carcinoma (29). The present invention also features fascin 2 as a polypeptide up-regulated in pancreatic cancer (SEQ ID NO: 56). Thus, a preferred embodiment of the present invention is a marker comprising SEQ ID. NO: 3. In another preferred embodiment, the marker comprises SEQ. ID NO: 58. In another preferred embodiment, the marker comprises SEQ. ID NO: 56.

In our study, cytokeratin 7 (SEQ. ID NO: 52) and cytokeratin 19 (SEQ ID NO: 33) showed strong expression in PC. Both have also been described in other cancers and have been linked with metastasis formation (Moll, R., Int. J. Biol. Markers, 9: 63-69, 1994.). High protein levels of actinin-4 (SEQ ID NO: 5) were detected in PC. This protein was linked by others with cell motility and cancer invasion (Honda, K., Yamada, T., Endo, R., Ino, Y., Gotoh, M., Tsuda, H., Yamada, Y., Chiba, H., and Hirohashi, S. J. Cell Biol., 140:1383-1393, 1998.). Taken together, the apparent strong expression of cytoskeletal proteins is likely to be an important factor in the strong invasiveness and metastasis-forming potential of PC. Thus, a preferred embodiment of the present invention is a marker comprising SEQ ID. NO: 52. In another preferred embodiment, the marker comprises SEQ. ID NO: 33. In another preferred embodiment, the marker comprises SEQ. ID NO: 5.

Metastasis

Cancer of exocrine pancreas is characterized by extensive local invasion, early lymphatic and hematogenous metastasis. Metastasis in PC has been found in the skeleton, eye, bladder, etc. The extent of angiogenesis depends on the balance between pro-angiogenic or anti-angiogenic factors released from cancer and host cell. Currently, intratumoral microvessel density (IMD) measured by immunocytochemistry appears to be the most reliable parameter for assessing angiogenic activity. Patients with high IMD have decreased survival rates in a variety of cancers (Fujioka, S., et al., Cancer, 92: 1788-1797, 2001). Thymidine phosphorylase (TYPH or TP, SEQ ID NO: 31) which is identical to platelet-derived endothelial cell growth factor, is strongly expressed in PC and stimulates the chemotaxis of endothelial cells through the 2-deoxy-D-ribose, degradation products of thymidine by TP, thus indirectly inducing angiogenesis (Haraguchi, M., et al. Nature, 368: 198, 1994.). Shuichi Fujioka et al. found that IMD and TP status were independent predictive indicators for overall as well as relapse-free survival in PC (Fujioka, S., et al., Cancer, 92: 1788-1797, 2001). An additional protein detected at higher levels in PC than in surrounding tissue likely involved in metastasis formation is osteoblast specific factor 2 (SEQ ID NO:53), a putative bone adhesion protein. Breast carcinoma commonly metastasizes to bone (Guise, T. A. Cancer, 88: 2892-2898, 2000). Although the role of this protein in PC is not clearly established, our findings suggest a similar role for osteoblast specific factor 2 in PC. Thus, a preferred embodiment of the present invention is a marker comprising SEQ ID. NO: 31. In another preferred embodiment, the marker comprises SEQ. ID NO: 53.

Small GTP-binding Proteins

Four small GTP-binding proteins and interacting proteins were more strongly expressed in PC than in normal pancreas tissue. These include RAN (SEQ. ID NO: 27), GBLP (guanine nucleotide binding protein β subunit-like protein RACK1, SEQ. ID NO: 47), GDIR (Rho GDP dissociation inhibitor 1, SEQ. ID NO: 55), and IQG1 or IQGAP1 (Ras gtpase activating like protein, SEQ ID NO: 25). Small GTP-binding proteins constitute a superfamily, which is structurally classified into at least five families: the Ras, Rho, Rab, Sar1/Arf, and Ran families and are involved in the regulation of gene expression, cytoskeletal reorganization, and nucleocytoplasmic transport (Takai, Y., et al. Physiol Rev., 81: 153-208, 2001). RAN is known to enhance androgen receptor-mediated transactivation and was shown to be overexpressed in prostate cancer (Sampson, E. R., et al., J. Biol. Regul. Homeost. Agents, 15: 123-129, 2001). Increased expression of RAN in 81% of prostate tumor cases, may contribute to over proliferation of prostate tumor cells (Li, P., et al., Am J Pathol., 161: 1467-1474,2002). GBLP is an anchoring protein for activated protein kinase Cβ and a variety of other proteins. Protein kinase C plays an important role in angiogenesis and cancer growth. Berns et al. found GBLP up-regulated in during angiogenesis in vitro and also associated with nonendothelial cells in angiogenically active tissue (Berns, H., et al., FASEB J., 14: 2549-2558, 2000). Further more, mRNA expression of GBLP is detected in epithelial cells of human colon carcinoma and proliferating epithelial cell of normal colon tissue. Therefore, there is a likely link between high GBLP expression and tumor growth. GDIR (Rho GDP dissociation inhibitor) had been found up-regulated in a chemoresistant fibrosarcoma cell line by 2D-PAGE (Sinha, P., et al., Electrophoresis, 20: 2961-2969, 1999) and may block apoptotic signal pathway mediated by Ras and c-jun kinase, resulting in the increase resistance against environmental stress. IQG1 (Ras GTPase-activating-like protein) is a widely expressed 190-kDa Cdc42-, Rac1-, and calmodulin-binding protein that interacts with F-actin in vivo and that can cross-link F-actin microfilaments in vitro. IQG1 negatively regulates the Ecc-based (E-cadherin/catenin complex) cell-cell adhesion by dissociating alpha-catenin. Up-regulation of IQGAP1 is correlated with the malignant phenotype in gastric cancer (Sugimoto, N., et al., J. Hum. Genet., 46: 21-25, 2001). By immunohistochemical analysis, IQGAP1 was found overexpressed in colorectal carcinoma and associated with carcinoma invasion (Nabeshima, K., et al., Cancer Lett., 176: 101-109, 2002). Since cancer invasiveness is associated with the localized disruption of cell-cell adhesion, both our results and Iacobuzio-Donahue et al.'s data suggest that IQGAP1 may be involved in the disruption of local adhesion and in PC invasion to surrounding tissue. Thus, a preferred embodiment of the present invention is a marker comprising SEQ ID NO: 27. In another preferred embodiment, the marker comprises SEQ. ID NO: 47. In another preferred embodiment, the marker comprises SEQ. ID NO: 55. In another preferred embodiment, the marker comprises SEQ. ID NO: 25.

S100 Protein Family

Another protein with high-level expression in PC is S109 (S100A9, MRP-14, calgranulin B, SEQ ID NO: 49), a member of the S100 protein family of highly homologous low molecular weight calcium binding proteins. Calgranulins are characterized by cell type-specific expression in cells of epithelial, myeloid and endothelial origin and accumulation at sites of acute and chronic inflammation (e.g. rheumatoid arthritis, cystic fibrosis, psoriasis, allergic dermatitis, inflammatory bowel diseases) (Donato, R. Int. J. Biochem. Cell Biol., 33: 637-668, 2001). S100A8 and S100A9 can form a noncovalent heterodimer protein complex called calprotectin. Current reports support that both of S100A9 and S100 A8 have wide range of possible intracellular as well as extracellular functions (Schafer, B. W. and Heizmann, C. W. Trends Biochem. Sci., 21: 134-140, 1996). S100A8 and S100A9 are negatively regulated by glucocorticoids in a c-Fos-dependent manner and over expressed throughout skin carcinogenesis (Gebhardt, C., et al., Oncogene, 21: 4266-4276, 2002). These proteins are also more strongly expressed in colorectal carcinoma than in matched normal colon mucosa, as shown by proteomics analysis (Stulik, J., et al., Electrophoresis, 20: 1047-1054, 1999). S10OA9 has been detected in cultured human adenocarcinoma (AC) cells derived from various organs, and is associated with tumor differentiation in pulmonary adenocarcinoma (Arai, K., et al., Oncol. Rep., 8: 591-596, 2001). Iacobuzio-Donahue et al's work indicates that over expression of S100A4 in PC is associated with poor differentiation and DNA hypomethylation (Rosty, C., et al., Am. J. Pathol., 160: 45-50, 2002). Thus, a preferred embodiment of the present invention is a marker comprising SEQ ID. NO: 49.

Annexin

We found that annexin 1 (SEQ ID NO: 51) and annexin 2 (SEQ ID NO: 19) have high level of expression in PC. Both are members of a family of Ca²⁺-dependent membrane-binding proteins. Described functions include, among others, an important role in malignant transformation (Masaki, T., et al., Hepatology, 24: 72-81, 1996), the control of epithelial cell line proliferation (Solito, E., et al., Cell Growth Differ., 9: 327-336, 1998), and mediation of apoptosis (Canaider, S., et al., Life Sci., 66: L265-L270, 2000). Evidence in support of causative roles for any annexins in the development of cancer is still mainly circumstantial. In MCF-7 breast carcinoma cells, overexpression of annexin1 led to abrogation of Ca2+ release after activation of purinergic or bradykinin receptors (Frey, B. M., et al., FASEB J., 13: 2235-2245, 1999), while over expression of annexin1 in rat 2 fibroblasts leads to direct inhibition of cytosolic PLA2, which in turn depresses the serum response element of c-fos (Oh, J., et al., FEBS Lett., 477: 244-248, 2000). Collectively, these studies imply a growth-suppressive role for annexin1. These results are not supported by the finding that annexin 1 is strongly up-regulated in a prostate cancer cell line (Vaarala, M. H., Lab Invest, 80: 1259-1268, 2000), esophageal cancer (Emmert-Buck, M. R., et al., Mol. Carcinog., 27: 158-165, 2000), a stomach cancer cell line (Sinha, P., et al., J. Biochem. Biophys. Methods, 37: 105-116, 1998), mammary adenocarcinoma (Pencil, S. D. and Toth, M. Clin. Exp. Metastasis, 16: 113-121, 1998), and hepatocarcinoma (de Coupade, C., et al., Hepatology, 31: 371-380, 2000). In hepatocarcinoma, study also showed that the proliferative rate of both normal and malignant hepatocytes was attenuated by antisense to annexin 1. These and our data suggest that cell growth is associated with elevated rather than reduced levels of annexin 1, which is also supported by the studies of Iacobuzio-Donahue et al. (Am. J. Pathol., 160: 1239-1249, 2002). Thus, a preferred embodiment of the present invention is a marker comprising SEQ ID NO: 51. In another preferred embodiment, the marker comprises SEQ. ID NO: 19.

Some additional proteins highly expressed in PC may have either clear roles in PC or an indirect link with PC, e.g. BGH3 (TGF-β1-induced protein, SEQ ID NO:6) which is a secretory protein and acts as a marker for biologically active TGF-β1 (Langham, R. G., et al., Transplantation, 72: 1826-1829, 2001). Thus, a preferred embodiment of the present invention is a marker comprising SEQ ID NO: 6.

With the identification of polypeptides regulated in pancreatic cancer, the present invention provides an in vitro method for the diagnosis of pancreatic cancer and/or the susceptibility to pancreatic cancer comprising the steps of obtaining a biological sample; and detecting and/or measuring the increase of a marker described hereinbefore.

The term “detection” as used herein refers to the qualitative determination of the absence or presence of polypeptides.

The term “measured” as used herein refers to the quantitative determination of the differences in expression of polypeptides in biological samples from patients with pancreatic cancer and biological samples from healthy individuals.

Methods for detection and/or measurement of polypeptides in biological samples are well known in the art and include, but are not limited to, Western-blotting, ELISAs or RIAs. Antibodies recognizing the polypeptides listed in table 2, 3, 5 and/or 6 can either be generated for the purpose of detecting the polypeptides, eg. by immunizing rabbits with purified proteins, or known antibodies recognizing the polypeptides can be used. For example, an antibody capable of binding to the denatured proteins, such as a polyclonal antibody, can be used to detect the peptides of this invention in a Western Blot. An example for a method to measure a marker is an ELISA. This type of protein quantitation is based on an antibody capable of capturing a specific antigen, and a second antibody capable of detecting the captured antigen. A further method for the detection of a diagnostic marker for pancreatic cancer is by analyzing biopsy specimens for the presence or absence of the markers of this invention. Methods for the detection of these markers are well known in the art and include, but are not limited to, immunohistochemistry or immunofluorescent detection of the presence or absence of the polypeptides of the marker of this invention. Methods for preparation and use of antibodies, and the assays mentioned hereinbefore are described in Harlow, E. and Lane, D. Antibodies: A Laboratory Manual, (1988), Cold Spring Harbor Laboratory Press.

The accuracy of the diagnosis of pancreatic cancer can be increased by analyzing combinations of multiple polypeptides listed in tables 2, 3, 5 and/or 6. Thus, the in vitro method herein before described, comprises a marker which comprises at least two, preferably at least three, more preferably at least four, even more preferably at least five, and most preferably at least six of the polypeptides listed in table 2,3, 5 and/or 6.

For diagnosis of pancreatic cancer, suitable biological samples need to be analyzed for the presence or absence of a marker. The biological samples can be serum, plasma, pancreatic juice or cells of pancreatic tissue. Cells from pancreatic tissue can be obtained by ERCP, secretin stimulation, fine-needle aspiration, cytologic brushings and large-bore needle biopsy.

It is also possible to diagnose pancreatic cancer by detecting and/or measuring nucleic acid molecules coding for the marker hereinbefore described. Preferably, the nucleic acid molecule is RNA or DNA. In another embodiment, the DNA is a cDNA.

In one embodiment of the present invention, the in vitro method herein before described comprises comparing the expression levels of at least two of the nucleic acids encoding the polypeptides in an individual suspected to suffer from pancreatic cancer and/or to be susceptible to pancreatic cancer, to the expression levels of the same nucleic acids in a healthy individual.

In another embodiment of the present invention the in vitro method herein before described comprises comparing the expression level of the marker in an individual suspected to suffer from pancreatic cancer and/or to be susceptible to pancreatic cancer to the expression levels of the same marker in a healthy individual. In a more preferred embodiment of the in vitro method, an increase or decrease of the expression levels of the marker is indicative of pancreatic cancer or the susceptibility to pancreatic cancer.

The present invention also provides a screening method for identifying and/or obtaining a compound which interacts with a polypeptide listed in table 2 and/or 3 whose expression is upregulated in pancreatic cancer, comprising the steps of contacting the polypeptide with a compound or a plurality of compounds under conditions which allow interaction of the compound with the polypeptide; and detecting the interaction between the compound or plurality of compounds with the polypeptide.

The “interaction” in the screening methods as disclosed herein may be measured by conventional methods. The type of conventional method for testing the interaction of a compound with a polypeptide that is soluble, as opposed to membrane associated, can be an in vitro method using either purified recombinant polypeptide, or native polypeptide purified from cells that endogenously express the polypeptide. As a non-limiting example, a polypeptide of the invention can be bound to beads or immobilized on plastic or other surfaces, and interaction of a compound with the polypeptide can be measured by either using a labeled compound and measuring the label bound to the polypeptide or by displacement of a labeled known ligand from the polypeptide.

For polypeptides that are associated with the cell membrane on the cell surface, or which are expressed as transmembrane or integral membrane polypeptides, the interaction of a compound with the polypeptides can be detected with different methods which include, but are not limited to, methods using cells that either normally express the polypeptide or in which the polypeptide is overexpressed, eg. by detecting displacement of a known ligand which is labeled by the compound to be screened. Alternatively, membrane preparations may be used to test for interaction of a compound with such a polypeptide

Interaction assays to be employed in the method disclosed herein may comprise FRET-assays (fluorescence resonance energy transfer; as described, inter alia, in Ng, Science 283 (1999), 2085-2089 or Ubarretxena-Belandia, Biochem. 38 (1999), 7398-7405), TR-FRETs and biochemical assays as disclosed herein. Furthermore, commercial assays like “Amplified Luminescent Proximity Homogenous Assay™” (BioSignal Packard) may be employed. Further methods are well known in the art and, inter alia, described in Fernandez, Curr. Opin. Chem. Biol. 2 (1998), 547-603.

The “test for interaction” may also be carried out by specific immunological and/or biochemical assays which are well known in the art and which comprise, e.g., homogenous and heterogenous assays as described herein below. The interaction assays employing read-out systems are well known in the art and comprise, inter alia, two-hybrid screenings (as, described, inter alia, in EP-0 963 376, WO 98/25947, WO 00/02911; and as exemplified in the appended examples), GST-pull-down columns, co-precipitation assays from cell extracts as described, inter alia, in Kasus-Jacobi, Oncogene 19 (2000), 2052-2059, “interaction-trap” systems (as described, inter alia, in U.S. Pat. No. 6,004,746) expression cloning (e.g. lamda gt11), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271 or WO 00/05410. Vidal and Legrain (1999) in Nucleic Acids Research 27, 919-929 describe, review and summarize further interaction assays known in the art which may be employed in accordance with the present invention.

Homogeneous (interaction) assays comprise assays wherein the binding partners remain in solution and comprise assays, like agglutination assays. Heterogeneous assays comprise assays like, inter alia, immuno assays, for example, Enzyme Linked Immunosorbent Assays (ELISA), Radioactive Immunoassays (RIA), Immuno Radiometric Assays (IRMA), Flow Injection Analysis (FIA), Flow Activated Cell Sorting (FACS), Chemiluminescent Immuno Assays (CLIA) or Electrogenerated Chemiluminescent (ECL) reporting.

The present invention further provides a screening method for identifying and/or obtaining a compound which is an inhibitor or an antagonist of a polypeptide listed in table 2 and/or 3 whose expression is upregulated in pancreatic cancer, comprising the steps of a) contacting the polypeptide with a compound identified and/or obtained by the screening method described above under conditions which allow interaction of the compound with the polypeptide; b) determining the activity of the polypeptide; c) determining the activity of the polypeptide expressed in the host as defined in (a), which has not been contacted with the compound; and d) quantitatively relating the activity as determined in (b) and (c), wherein a decreased activity determined in (b) in comparison to (c) is indicative for an inhibitor or antagonist. The terms inhibitors and antagonists as used herein are used interchangeably. This screening assay can be performed either as an in vitro assay, or as a host-based assay. The host to be employed in the screening methods of the present invention and comprising and/or expressing a polypeptide listed in table 2, 3, 5 and/or 6 may comprise prokaryotic as well as eukaryotic cells. The cells may comprise bacterial cells, yeast cells, as well as cultured (tissue) cell lines, inter alia, derived from mammals. Furthermore animals may also be employed as hosts, for example an non-human transgenic animal. Accordingly, the host (cell) may be transfected or transformed with the vector comprising a nucleic acid molecule coding for a polypeptide which is differentially regulated in pancreatic cancer as disclosed herein. The host cell or host may therefore be genetically modified with a nucleic acid molecule encoding such a polypeptide or with a vector comprising such a nucleic acid molecule.

The term “genetically modified” means that the host cell or host comprises in addition to its natural genome a nucleic acid molecule or vector coding for a polypeptide listed in table 2, 3, 5 and/or 6 or at least a fragment thereof. The additional genetic material may be introduced into the host (cell) or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host.

As mentioned herein above, the host cell of the present invention may be any prokaryotic or eukaryotic cell. Suitable prokaryotic cells are those generally used for cloning like E. coli or Bacillus subtilis. Yet, these prokaryotic host cells are also envisaged in the screening methods disclosed herein. Furthermore, eukaryotic cells comprise, for example, fungal or animal cells. Examples for suitable fungal cells are yeast cells, preferably those of the genus Saccharomyces and most preferably those of the species Saccharomyces cerevisiae. Suitable animal cells are, for instance, insect cells, vertebrate cells, preferably mammalian cells, such as e.g. CHO, HeLa, NIH3T3 or MOLT-4. Further suitable cell lines known in the art are obtainable from cell line depositories, like the American Type Culture Collection (ATCC).

The hosts may also be selected from non-human mammals, most preferably mice, rats, sheep, calves, dogs, monkeys or apes. As described herein above, the animals/mammals also comprise non-human transgenic animals, which preferably express at least one polypeptide differentially regulated in pancreatic cancer as disclosed herein. Preferably, the polypeptide is a polypeptide which is up-regulated in tissue derived from patients with pancreatic cancer. Yet it is also envisaged that non-human transgenic animals be produced which do not express marker genes as disclosed herein or who express limited amounts of the marker gene products. The animals are preferably related to polypeptides which are down-regulated in pancreatic cancer. Transgenic non-human animals comprising and/or expressing the up-regulated polypeptides of the present invention or alternatively, which comprise silenced or less efficient versions of down-regulated polypeptides are useful models for studying the development of pancreatic cancer and provide for useful models for testing drugs and therapeutics for pancreatic cancer treatment and/or prevention.

A compound which interacts with a polypeptide listed in table 2, 3, 5 and/or 6 and which inhibits or antagonizes the polypeptide is identified by determining the activity of the polypeptide in the presence of the compound.

The term “activity” as used herein relates to the functional property or properties of a specific polypeptide. For the enzymes listed in table 2, 3, 5 and/or 6, the term “activity” relates to the enzymatic activity of a specific polypeptide. Activity assays for the enzymes listed in table 2, 3, 5 and/or 6 are well known.

For adhesion molecules listed in table 2, 3, 5 and/or 6, the term “activity” relates to the adhesive properties of a polypeptide and may be determined using assays such as, but not limited to, adhesion assays, cell spreading assays, or in vitro interaction of the adhesion molecule with a known ligand. Such assays are well known in the art.

For cytoskeletal proteins, the term “activity” relates to the regulation of the cytoskeleton by such polypeptides, or to their incorporation into the cytoskeleton. As a non-limiting example, the ability of Gelsolin to regulate actin polymerization, or of Filamin A to promote orthogonal branching of actin filaments, may be determined using in vitro actin polymerization assays. Activity in relation to the regulation of cytoskeletal structures may further be determined by, as non-limiting examples, cell spreading assays, cell migration assays, cell proliferation assays or immunofluorecence assays, or by staining actin filaments with fluorescently labeled phalloidin. All of these assays are well known to the person skilled in the art.

For ion channels (Chloride intracellular channel protein) the term “activity” relates to ion flux (Chloride lux) across the membrane. Methods to determine ion flux across membranes are well known to the person skilled in the art.

For transcription factors, eg. KIAA 1034, the term “activity” relates to their ability to regulate gene transcription. The transcriptional activity of a polypeptide can be determined using commonly used assays, such as a reporter gene assay.

For growth factors and hormones or their receptors, the term “activity” relates to their ability to bind to their receptors or ligands, respectively, and to induce receptor activation and subsequent signaling cascades, and/or it relates to the factor's or receptor's ability to mediate the cellular function or functions eventually caused by growth factor or hormone mediated receptor activation. Growth factor or hormone binding to receptors can be determined by commonly known ligand binding assays. Receptor activation can be determined by testing for receptor auto-phosphorylation, or by assaying for modification or recruitment of downstream signaling mediators to the receptors (by immunoprecipitation and Western Blotting of signaling complexes). Cellular functions regulated by growth factors or hormones and their receptors can be cell proliferation (eg determined by using thymidine incorporation or cell counts), cell migration assays (eg determined by using modified Boyden chambers), cell survival or apoptosis assays (eg determined by using DAPI staining), angiogenesis assays (eg in vitro assays to measure endothelial tube formation that are commercially available). In addition to these assays, other assays may be used as well to determine these and other cellular functions.

Inhibitors or antagonists of a polypeptide listed in tables 2 and/or 3 are identified by the screening method described above when there is a decreased activity determined in the presence of the compound in comparison to the absence of the compound in the screening method, which is indicative for an inhibitor or antagonist.

Further to the screening methods disclosed above, this invention provides a screening method for identifying and/or obtaining a compound which is an inhibitor of the expression of a polypeptide listed in tables 2 and/or 3 whose expression is upregulated in pancreatic cancer, comprising the steps of a) contacting a host which expresses the polypeptide with a compound; b) determining the expression level and/or activity of the polypeptide; c) determining the expression level and/or activity of the polypeptide in the host as defined in (a), which has not been contacted with the compound; and d) quantitatively relating the expression level of the polypeptide as determined in (b) and (c), wherein a decreased expression level determined in (b) in comparison to (c) is indicative for an inhibitor of the expression of the polypeptide.

An inhibitor of the expression of a polypeptide listed in table 2, 3, 5 and/or 6 is identified by the screening method described hereinbefore when a decreased expression of the protein is determined in the presence of the compound in comparison to the absence of the compound in the screening method, which is indicative for an inhibitor of expression of a polypeptide.

The term “express” as used herein relates to expression levels of a polypeptide listed in table 2, 3, 5 and/or 6 which is up-regulated in pancreatic cancer, in cells, preferably in a pancreatic adenocarcinoma cell line, which are elevated as compared to the expression levels of the same polypeptide in healthy pancreatic cells. Preferably, expression levels are at least 2 fold, more preferably at least 3 fold, even more preferably at least 4 fold, most preferably at least 5 fold higher than in healthy pancreatic cells.

Furthermore, the present invention provides a compound identified and/or obtained by any of the screening methods hereinbefore described. The compound is further comprised in a pharmaceutical composition. A method for the preparation of the pharmaceutical composition comprising formulating the compound in a pharmaceutically acceptable carrier or diluent is also claimed. Any conventional carrier material can be utilized. The carrier material can be an organic or inorganic one suitable for eteral, percutaneous or parenteral administration. Suitable carriers include water, gelatin, gum arabic, lactose, starch, magnesium stearate, talc, vegetable oils, polyalkylene-glycols, petroleum jelly and the like. Furthermore, the pharmaceutical preparations may contain other pharmaceutically active agents. Additional additives such as flavoring agents, stabilizers, emulsifying agents, buffers and the like may be added in accordance with accepted practices of pharmaceutical compounding.

The compound may be used for the preparation of a medicament for the treatment or prevention of pancreatic cancer. In addition, the compound may also be used for the preparation of a diagnostic composition for diagnosing pancreatic cancer or a predisposition for pancreatic cancer. Preferably, the compound comprises an antibody, an antibody-derivative, an antibody fragment, a peptide or an antisense construct.

Within the scope of the present invention, antibodies against the proteins listed in tables 2 and/or 3, or antigen-binding fragments thereof, may be used in an in vitro method for the diagnosis of pancreatic cancer.

In order to efficiently perform diagnostic screenings, the present invention provides a kit for the diagnosis of pancreatic cancer comprising one or more of the antibodies, or antigen-binding fragments thereof, described above. Another kit provided by this invention is a kit for the diagnosis of pancreatic cancer comprising one or more of the nucleic acids coding for the marker hereinbefore described. Yet another kit provided by this invention is a kit for screening of compounds that antagonize any of the polypeptides listed in tables 2 and/or 3 or inhibit the expression of any of the polypeptides.

The present invention pertains to a marker for diagnosis of pancreatic cancer comprising at least one polypeptide selected from the group consisting of the polypeptides listed in table 6. Preferably, the marker does not include SEQ ID NO:s 25 and 50 to 55. In a more preferred embodiment, the marker comprises at least one of the polypeptides listed in table 5.

The present invention also provides an in vitro method for the diagnosis of pancreatic cancer and/or the susceptibility to pancreatic cancer comprising the steps of

a) obtaining a biological sample; and

b) detecting and/or measuring the increase of at least one of the polypeptides listed in table 6.

Preferably, the in vitro method additionally comprises the step of detecting and/or measuring the decrease of at least one of the polypeptides listed in table 5. More preferably, in the in vitro method, the at least one polypeptide does not include SEQ ID NO:s 25 and 50 to 55. Even more preferably, in the in vitro method, the biological sample is derived from the group consisting of serum, plasma, pancreatic juice and cells of pancreatic tissue.

The present invention further provides an in vitro method for the diagnosis of pancreatic cancer and/or the susceptibility to pancreatic cancer comprising the steps of

a) obtaining a biological sample; and

b) detecting and/or measuring the increase of at least one nucleic acid coding for the marker hereinbefore described.

Preferably, in the in vitro method, the nucleic acid molecule is RNA or DNA. More preferably, in the in vitro method, the DNA is a cDNA.

In a further more preferred embodiment of any of the in vitro methods hereinbefore described, the expression levels of at least one of the nucleic acids in an individual suspected to suffer from pancreatic cancer and/or to be susceptible to pancreatic cancer is compared to the expression levels of the same nucleic acids in a healthy individual. In a most preferred embodiment of any of the in vitro methods hereinbefore described, the expression level of the marker in an individual suspected to suffer from pancreatic cancer and/or to be susceptible to pancreatic cancer is compared to the expression levels of the same marker in a healthy individual.

In a further preferred embodiment of the in vitro method hereinbefore described, an increase of the expression levels of the marker is indicative of pancreatic cancer or the susceptibility to pancreatic cancer.

The present invention also pertains to a screening method for identifying and/or obtaining a compound which interacts with a polypeptide selected from the group consisting of the polypeptides listed in table 6 whose expression is upregulated in pancreatic cancer, comprising the steps of

a) contacting the polypeptide with a compound or a plurality of compounds under conditions which allow interaction of the compound with the polypeptide; and

b) detecting the interaction between the compound or plurality of compounds with the polypeptide.

Furthermore, the present invention provides a screening method for identifying and/or obtaining a compound which is an inhibitor or an antagonist of a polypeptide listed in table 6 whose expression is upregulated in pancreatic cancer, comprising the steps of

a) contacting the polypeptide with a compound identified and/or obtained by the screening method of claim 39 under conditions which allow interaction of the compound with the polypeptide;

b) determining the activity of the polypeptide;

c) determining the activity of the polypeptide expressed in the host as defined in (a), which has not been contacted with the compound; and

d) quantitatively relating the activity as determined in (b) and (c), wherein a decreased activity determined in (b) in comparison to (c) is indicative for an inhibitor or antagonist.

The present invention also provides a screening method for identifying and/or obtaining a compound which is an inhibitor of the expression of a polypeptide selected from the group consisting of the polypeptides listed in table 6 whose expression is upregulated in pancreatic cancer, comprising the steps of

a) contacting a host which expresses the polypeptide with a compound,

b) determining the expression level and/or activity of the polypeptide;

c) determining the expression level and/or activity of the polypeptide in the host as defined in (a), which has not been contacted with the compound; and

d) quantitatively relating the expression level of the polypeptide as determined in (b) and (c), wherein a decreased expression level determined in (b) in comparison to (c) is indicative for an inhibitor of the expression of the polypeptide.

The present invention provides a compound identified and/or obtained by the screening methods hereinbefore described.

In addition, the present invention provides a pharmaceutical composition comprising the compound hereinbefore described. Also provided is a method for the preparation of the pharmaceutical composition hereinbefore described comprising formulating the compound hereinbefore described in a pharmaceutically acceptable carrier or diluent.

The present invention provides a use of a compound hereinbefore described for the preparation of a medicament for the treatment or prevention of pancreatic cancer. Also provided is a use of a compound hereinbefore described for the preparation of a diagnostic composition for diagnosing pancreatic cancer or a predisposition for pancreatic cancer. In a preferred embodiment, the uses hereinbefore described relate to a compound comprising an antibody, an antibody-derivative, an antibody fragment, a peptide or an antisense construct.

Within the scope of the present invention, antibodies against the proteins listed in tables 5 and/or 6, or antigen-binding fragments thereof, may be used in an in vitro method for the diagnosis of pancreatic cancer.

In order to efficiently perform diagnostic screenings, the present invention provides a kit for the diagnosis of pancreatic cancer comprising one or more of the antibodies, or antigen-binding fragments thereof, described above. Another kit provided by this invention is a kit for the diagnosis of pancreatic cancer comprising one or more of the nucleic acids coding for the marker hereinbefore described. Yet another kit provided by this invention is a kit for screening of compounds that antagonize any of the polypeptides listed in tables 5 and/or 6 or inhibit the expression of any of the polypeptides.

In the present invention, the proteins, compounds, kits, methods and uses substantially as herein before described, especially with reference to the foregoing examples are also claimed.

### EXAMPLES

Collection of Tissue Samples

Pancreatic carcinomas and adjacent tissue were collected from the patients listed in table 1. Samples were collected shortly after the resection (less than 30 minutes), and fast frozen in liquid nitrogen for about 1 minute, then stored in a freezer at a temperature of −80° C.

Characterization of Formalin-fixed Specimens

Histopathological characterization was carried out by using hematoxylin-eosin-stained sections of formalin-fixed and paraffin-embedded specimens. Tumors were classified using the WHO system. The types of pancreatic carcinomas included in the study are shown in table 1.

The twelve pancreatic carcinoma samples used in this study were ductal carcinomas which constitute the overwhelming proportion of pancreatic carcinomas. The patient-matched samples from histologically normal tissue surrounding the carcinoma were used as controls. We carried out 12 pairs of 2-dimensional electrophoresis maps for comparing protein expression between tumor tissue and normal control tissue. For protein identification, the samples were pooled, thus generating pan-Carcinoma and pan-Normal protein extracts. Quantification was carried out in two steps: (I) Gels from the pooled samples were compared using the PDQuest image analysis software. (II) The changes identified at the level of the pooled samples were cross-validated by an analysis of the individual samples. The change factors shown in table 2, 3, 5 and 6 were determined using the pooled samples.

Preparation of Samples for Electrophoresis

Samples cleaned of clots and contaminating tissue were frozen in liquid nitrogen, then ground to powder. Samples were suspended in lysis buffer (8M urea, 4% CHAPS, 40 mMol/L Tris-Cl, 0.5% carrier amphollytes, 100 mMol/L DTT and 0.1 ig/il PMSF) and centrifuged at 12000 rpm for 30 minutes. The supernatants were stored at −80° C. The protein concentration in the extracts was determined by the Bradford method (Bradford, M. Anal. Biochem. 72, 248 (1976).

Two-dimensional Gel Electrophoresis

Samples containing 1 mg of protein were loaded onto the rehydrated IPG strip (18 cm, pH3˜10) by using the cup loading method. IEF was performed using Pharmacia Multiphor apparatuses under the following conditions: First, the voltage was increased 200V-5000V over 24 hrs, then a constant voltage of 5000V was applied for 24 hrs, the running temperature was 20° C. After IEF, the strips were equilibrated with 10 ml equilibration solution I (6 M Urea, 50 mM Tris pH 8.8, 30% Glycerol, 2.0% SDS, 30 mM Dithioerythritol) for 15 min, then for another 15 min with equilibration solution II (6 M Urea, 50 mM Tris pH 8.8, 30% Glycerol, 2.0% SDS, 0.23 M Iodoacetamide).

The second dimension SDS polyacrylamide gel electrophoresis (SDS-PAGE) was carried out using a Hoefer ISO_DALT apparatus (10 gels/run, 24×20 cm), IEF strips were loaded onto 12% homogeneous polyacrylamide gels (1.5 mm×24 cm×20 cm). The gels were run in TGS_Buffer (250 mM Tris, 1.92 M Glycine, 1% (w/v) SDS, pH=8.3, Bio-Rad) at a constant voltage (80 V, 20° C.).

Gel Fixation and Staining

Gels were fixed in 50% Methanol/20% acetic acid for 30 min, then washed in ultra-pure water for 30 min and stained with NOVEX Colloidal Blue staining Kit (Invitrogen) following the manufacturer's recommendations.

Protein Identification

The protein identification was performed using a two-step procedure.

In-gel Digestion

Spots were picked and transferred into 96-well by a spot picking robot. From each gel, 600-800 spots were picked. The spots were destained with 100 μl of 30% acentonitrile in 50 Mm ammonium bicarbonate, washed in ultra pure-water and dried in a speed vac evaporator. The dry gel pieces were digested with 10 ng/μl trypsin (Promega, Madison, USA) solution in 500 nM ammonium bicarbonate at room temperature for 16 h maximum. The peptides from each spot were extracted with 20 μl of 0.1% trifluore acetic acid (TFA) in 50% acetonitrile. The matrix solution consisted of 0.025%(w/v) alfa-cyano-4-hydroxy cinammic acid (Sigma) in 50% acetonitrile/0.1% TFA with internal standard peptides des-Arg-Bradykinin (Sigma, MW 904.4681 Da) and adrenocorticotropic hormone fragment 18-39 (Sigma, MW 2465.1989 Da).

Analysis by MALDI-TOF

1.5 ul of peptide extract and 1.0 μl of matrix solution were stimulaneously applied to the spots on the MS target. Recrystallization was carried out as specified by the instruments manufacturer. The samples were analyzed in a MALDI-time of flight Mass spectrometer (Autoflex, Bruker Analytics, Bremen, Germany). Peak annotation and database search by peptide matching was performed by in house developed software. The peptide mass was compared with theoretic peptide masses of all available proteins from all species. The monoisotopic mass was used and a mass tolerance of 0.0025% was allowed. 4 matching peptides were the minimal requirement for an identity assignment. Mismatch or miscleavage sites were not considered.

TABLE 1
Clinical and histopathological characteristics of samples

| | | | | | Metas- |
|---|---|---|---|---|---|
| | | | | | tasis in |
| NO: of | | | Tumor | | lymph |
| Samples | Sex | Age | location | Histology | nodes |
| PC-01 | Male | 48 | Head of | Middle differentiated | Yes |
| | | | pancreas | ductal adenocarcinoma | |
| PC-02 | Male | 68 | Head of | Poorly differentiated | Yes |
| | | | pancreas | adenocarcinoma | |
| PC-03 | Male | 44 | Head of | Poorly differentiated | Yes |
| | | | pancreas | ductal adenocarinoma, | |
| | | | | clear cell type | |
| PC-04 | Male | 66 | Head of | Well differentiated ductal | Yes |
| | | | pancreas | adenocarcinoma | |
| PC-05 | Female | 45 | Head of | Well differentiated ductal | No |
| | | | pancreas | adenocarcinoma | |
| PC-06 | Female | 65 | Head of | Well differentiated ductal | Yes |
| | | | pancreas | adenocarcinoma | |
| PC-07 | Male | 59 | Head of | Middle differentiated | Yes |
| | | | pancreas | ductal adenocarcinoma | |
| PC-08 | Female | 62 | Body of | Well differentiated ductal | Yes |
| | | | pancreas | adenocarcinoma | |
| PC-09 | Male | 54 | Head of | Middle differentiated | No |
| | | | pancreas | ductal adenocarcinoma | |
| PC-10 | Female | 53 | Head of | Well differentiated ductal | No |
| | | | pancreas | adenocarcinoma | |
| PC-11 | Female | 54 | Head of | Middle differentiated | Yes |
| | | | pancreas | ductal adenocarcinoma | |
| PC-12 | Female | 69 | Head of | Middle differentiated | Yes |
| | | | pancreas | ductal adenocarcinoma | |

TABLE 2
Proteins up-regulated in pancreatic cancer I

| | | | SEQ ID | Fold |
|---|---|---|---|---|
| Protein | Acc No | Description | NO: | Change |
| sw:CATD_HUMAN | P07339 | Cathepsin D precursor (ec 3.4.23.5). | 1 | <2 |
| sw:IDHC_HUMAN | O75874 | Isocitrate dehydrogenase [NADP] cytoplasmic | 2 | 2 |
| | | (ec 1.1.1.42) | | |
| sw:GELS_HUMAN | P06396 | Gelsolin precursor, plasma | 3 | 3 |
| sw:CFAB_HUMAN | P00751 | Complement factor B precursor (ec 3.4.21.47) | 4 | 5 |
| sw:AAC4_HUMAN | O43707 | Alpha-actinin 4 (non-muscle alpha-actinin 4) | 5 | 2 |
| sw:AAC1_HUMAN | P12814 | Alpha-actinin 1 (alpha-actinin cytoskeletal | 7 | 2 |
| | | isoform) | | |
| sw:TBA4_HUMAN | P05215 | Tubulin alpha-4 chain. | 8 | 2 |
| sw:ABP2_HUMAN | P21333 | Filamin A (Endothelial actin-binding protein) | 9 | 4 |
| sw:TAGL_HUMAN | P37802 | Transgelin 2 (smooth muscle protein 22-alpha) | 10 | 2 |
| sw:TPM4_HUMAN | P07226 | Tropomyosin alpha 4 chain | 11 | <2 |
| sw:BGH3_HUMAN | Q15582 | Transforming growth factor-beta induced | 6 | 5 |
| | | protein IG-H3 precursor | | |
| sw:CALD_HUMAN | Q05682 | Caldesmon (cdm) | 12 | 2 |
| sw:ENOL_HUMAN | Q05524 | Alpha enolase | 13 | 2 |
| sw:ACY1_HUMAN | Q03154 | Aminoacylase-1 | 14 | 5 |
| sw:CAPB_HUMAN | P47756 | F-actin capping protein beta subunit (capz beta) | 15 | 5 |
| sw:IPYR_HUMAN | Q15181 | Inorganic pyrophosphatase | 16 | <2 |
| sw:LEG3_HUMAN | P17931 | Galectin-3 (galactose-specific lectin 3). | 17 | 2 |
| sw:POR2_HUMAN | P45880 | Voltage-dependent anion-selective channel | 18 | <2 |
| | | protein 2 | | |
| SW:ANX2_HUMAN | P07355 | Annexin II | 19 | 2 |
| sw:CBP2_HUMAN | P50454 | Collagen-binding protein 2 precursor | 20 | 2 |
| sw:COF1_HUMAN | P23528 | Cofilin, non-muscle isoform | 21 | <2 |
| sw:CYPH_HUMAN | P05092 | Peptidyl-prolyl cis-trans isomerase A | 22 | <2 |
| sw:DYI2_HUMAN | Q13409 | Dynein intermediate chain 2, cytosolic | 23 | 2 |
| sw:ECH1_HUMAN | Q13011 | Delta3,5-Delta2,4-dienoyl-coa isomerase, | 24 | 2 |
| | | mitochondrial precursor | | |
| sw:MLRN_HUMAN | P24844 | Myosin regulatory light chain 2 | 48 | 2 |
| sw:PLSL_HUMAN | P13796 | L-Plastin | 26 | <2 |
| sw:RAN_HUMAN | P17080 | GTP-binding nuclear protein ran | 27 | 3 |
| sw:ROK_HUMAN | Q07244 | Heterogeneous nuclear ribonucleoprotein k | 28 | 2 |
| sw:TCTP_HUMAN | P13693 | Translationally controlled tumor | 29 | <2 |
| sw:TPM1_HUMAN | P09493 | Tropomyosin 1 alpha chain | 30 | <2 |
| sw:TYPH_HUMAN | P19971 | Thymidine phosphorylase precursor | 31 | 5 |
| sw:AMPL_HUMAN | P28838 | Cytosol aminopeptidase | 32 | 3 |
| sw:K1CS_HUMAN | P08727 | Keratin, type I cytoskeletal 19 (cytokeratin 19) | 33 | 4 |
| sw:ALDX_HUMAN | P14550 | Alcohol dehydrogenase [NADP+] | 34 | 4 |
| sw:EL3A_HUMAN | P09093 | Elastase IIIa precursor | 35 | 4 |
| sw:DLDH_HUMAN | P09622 | Dihydrolipoamide dehydrogenase, | 36 | 2 |
| | | mitochondrial precursor | | |
| sw:ECHM_HUMAN | P30084 | Enoyl-CoA hydratase, mitochondrial precursor | 37 | 3 |
| sw:HSBX_HUMAN | O14558 | Heat-shock 20 kDa like-protein p20. | 38 | 2 |
| sw:MLEN_HUMAN | P16475 | Myosin light chain alkali, non-muscle isoform | 39 | 3 |
| sw:CALX_HUMAN | P27824 | Calnexin precursor | 40 | 3 |
| sw:MA32_HUMAN | Q07021 | Complement component 1 | 41 | <2 |
| sw:NUAM_HUMAN | P28331 | NADH-ubiquinone oxidoreductase 75 kda | 42 | 2 |
| | | subunit, mitochondrial precursor | | |
| sw:PBEF_HUMAN | P43490 | Pre-B cell enhancing factor precursor. | 43 | 2 |
| sw:RET1_HUMAN | P09455 | Retinol-binding protein I, cellular | 44 | 2 |
| sw:TCPG_HUMAN | P49368 | T-complex protein 1, gamma subunit | 45 | 2 |
| sw:RINI_HUMAN | P13489 | Placental ribonuclease inhibitor | 46 | <2 |
| sw:GBLP_HUMAN | P25388 | Guanine nucleotide-binding protein beta | 47 | 2 |
| | | subunit-like protein 12.3 | | |
| sw:S109_HUMAN | P06702 | Calgranulin B | 49 | <2 |

TABLE 3
Proteins up-regulated in pancreatic cancer II

| | | | SEQ ID | Fold |
|---|---|---|---|---|
| Protein | Acc No | Description | No | Change |
| sw:CAPG_HUMAN | P40121 | Macrophage capping protein | 50 | 3 |
| sw:ANX1_HUMAN | P04083 | Annexin I (lipocortin I) (calpactin II) | 51 | 4 |
| sw:K2C7_HUMAN | P08729 | Keratin, type II cytoskeletal 7 | 52 | 5 |
| humangp:CHR13- | Q15063 | Osteoblast specific factor 2 precursor | 53 | 2 |
| Q15063 | | | | |
| sw:TGLC_HUMAN | P21980 | Protein-glutamine gamma-glutamyltransferase | 54 | 2 |
| sw:GDIR_HUMAN | P52565 | Rho GDP-dissociation inhibitor 1 | 55 | <2 |
| sw:IQG1_HUMAN | P46940 | Ras GTPase-activating-like protein | 25 | 2 |

TABLE 4
Proteins roughly classified by their involved
biological processing or basic function

| Protein function^{a} | Number^{b} | Percentage (%)^{c} |
|---|---|---|
| Structural constitural and regulation | 61 | 20.8 |
| of cytoskeleton | | |
| Cell cycle and metabolism | 74 | 25.3 |
| Response to external stimulus or | 61 | 20.8 |
| stress | | |
| Signal transduction | 13 | 4.4 |
| Nuclear function | 18 | 6.1 |
| Transport processing | 19 | 6.5 |
| Hemostatis | 12 | 4.1 |
| Cell adhension | 7 | 2.4 |
| Chaperon | 7 | 2.4 |
| Apoptosis | 3 | 1 |
| Unknown function | 11 | 3.7 |
| Others | 7 | 2.4 |
| Total | 293 | 100 |

TABLE 5
Proteins with higher levels in normal pancreatic compared to cancer tissue

| | | | | SEQ ID |
|---|---|---|---|---|
| Protein^{a} | AccNo^{b} | Description^{c} | Fold^{d} | NO: |
| cytoskeletal | | | | |
| regulation | | | | |
| sw:DESM_HUMAN | P17661 | desmin. | 3 | 70 |
| proteolysis and | | | | |
| peptidolysis | | | | |
| sw:CBPB_HUMAN | P15086 | carboxypeptidase b precursor | 2 | 71 |
| sw:CBP1_HUMAN | P15085 | carboxypeptidase a1 precursor | Uncalculated* | 72 |
| sw:CPB2_HUMAN | P50454 | carboxypeptidase a2 precursor | Uncalculated* | 73 |
| sw:CTRB_HUMAN | P17538 | chymotrypsinogen b precursor | Uncalculated* | 74 |
| sw:TRY1_HUMAN | P07477 | trypsin I precursor (ec | Uncalculated* | 75 |
| | | 3.4.21.4) (cationic | | |
| | | trypsinogen). | | |
| sw:TRY2_HUMAN | P07478 | trypsin ii precursor (anionic | 3 | 76 |
| | | trypsinogen). | | |
| sw:ILEU_HUMAN | P30740 | leukocyte elastase inhibitor | 4 | 77 |
| chaperon | | | | |
| sw:CH60_HUMAN | P10809 | human. Mitochondrial matrix | 2 | 78 |
| | | protein p1 precursor | | |
| sw:ENPL_HUMAN | P14625 | 94 kda glucose-regulated | 3 | 79 |
| | | protein | | |
| SW:ER29_HUMAN | P30040 | endoplasmic reticulum protein | 2 | 80 |
| | | erp29 precursor | | |
| sw:PDA2_HUMAN | Q13087 | protein disulfide isomerase a2 | 2 | 81 |
| | | precursor | | |
| sw:PDA3_HUMAN | P30101 | protein disulfide isomerase a3 | 2 | 82 |
| | | precursor | | |
| oxidoreductase | | | | |
| sw:ADHB_HUMAN | P00325 | alcohol dehydrogenase beta | <2 | 83 |
| | | chain | | |
| sw:GTO1_HUMAN | P78417 | glutathione transferase omega1 | Uncalculated* | 84 |
| sw:OXRP_HUMAN | Q9Y4L1 | 150 kda oxygen-regulated | Uncalculated* | 85 |
| | | protein precursor | | |
| sw:PDX4_HUMAN | Q13162 | peroxiredoxin 4 | <2 | 86 |
| sw:ULA4_HUMAN | P30039 | mawd binding protein | <2 | 87 |
| metabolism of | | | | |
| biological process | | | | |
| sw:AMYC_HUMAN | P19961 | alpha-amylase 2b precursor | 3 | 88 |
| sw:AMYP_HUMAN | P04746 | alpha-amylase, pancreatic | Uncalculated* | 89 |
| | | precursor | | |
| sw:ATPA_HUMAN | P25705 | atp synthase alpha chain, | <2 | 90 |
| | | mitochondrial precursor | | |
| sw:BAL_HUMAN | P19835 | bile-salt-activated lipase | 3 | 91 |
| | | precursor | | |
| sw:LIP1_HUMAN | P54315 | pancreatic lipase related | <2 | 92 |
| | | protein 1 precursor. | | |
| sw:LIP2_HUMAN | P54317 | pancreatic lipase related | Uncalculated* | 93 |
| | | protein 2 precursor | | |
| sw:LIPP_HUMAN | P16233 | triacylglycerol lipase, | Uncalculated* | 94 |
| | | pancreatic precursor | | |
| sw:DPY2_HUMAN | Q16555 | dihydropyrimidinase related | | 95 |
| | | protein-2 | | |
| sw:GABT_HUMAN | P80404 | 4-aminobutyrate | 2 | 96 |
| | | aminotransferase | | |
| sw:GATM_HUMAN | P50440 | glycine amidinotransferase, | Uncalculated* | 97 |
| | | mitochondrial precursor | | |
| sw:GR78_HUMAN | P11021 | 78 kda glucose-regulated | 3 | 98 |
| | | protein precursor (grp 78) | | |
| sw:IF32_HUMAN | Q13347 | eukaryotic translation | <2 | 99 |
| | | initiation factor 3 subunit 2 | | |
| sw:DPY2_HUMAN | Q16555 | dihydropyrimidinase related | | 100 |
| | | protein-2 | | |
| sw:PGMU_HUMAN | P36871 | phosphoglucomutase | 2 | 101 |
| sw:PSA1_HUMAN | P25786 | proteasome subunit alpha type 1 | 2 | 102 |
| heat shock protein | | | | |
| sw:HS27_HUMAN | P04792 | heat shock 27 kda protein | Uncalculated* | 103 |
| signaling | | | | |
| SW:PD6I | Q8WUM4 | programmed cell death 6 | 5 | 104 |
| | | interacting protein | | |
| muscle development | | | | |
| hsugp:057687-10-0 | Q9bva2 | four and a half lim domains 3 | <2 | 105 |
| SW:sli2 | Q13643 | Skeletal muscle LIM-protein 2 | <2 | 106 |
| | | (SLIM 2)(Four and a half LIM | | |
| | | domains protein 3)(FHL.3). | | |
| transport of | | | | |
| biological process | | | | |
| sw:CYB5_HUMAN | P00167 | cytochrome b5. | 2 | 107 |
| cell adhesion | | | | |
| sw:PAP1_HUMAN | Q06141 | pancreatitis-associated protein | Uncalculated* | 108 |
| | | 1 precursor. | | |
| other | | | | |
| SW:CA16_HUMAN | P12109 | collagen alpha 1(vi) chain | 5 | 109 |
| | | precursor. | | |
| SW:LUM_HUMAN | P51884 | lumican precursor | 3 | 110 |

TABLE 6
Proteins with higher levels in pancreatic cancer compared to in normal tissue

| | | | | SEQ ID |
|---|---|---|---|---|
| Protein^{a} | AccNo^{b} | Description^{c} | Fold^{d} | NO: |
| cytoskeletal regulation | | | | |
| sw:FSC2_HUMAN | O14926 | fascin 2 (retinal fascin). | 2 | 56 |
| sw:AAC1_HUMAN | P12814 | alpha-actinin 1 | 2 | 7 |
| sw:AAC4_HUMAN | O43707 | alpha-actinin 4 | 2 | 5 |
| sw:ABP2_HUMAN | P21333 | endothelial actin-binding protein | 4 | 9 |
| | | (alpha-filamin). | | |
| sw:ANX2_HUMAN* | P07355 | human annexin ii (lipocortin ii) | 2 | 19 |
| sw:CALD_HUMAN | Q05682 | caldesmon (cdm). | 2 | 12 |
| sw:CAPB_HUMAN | P47756 | f-actin capping protein beta subunit | 5 | 15 |
| sw:CAPG_HUMAN* | P40121 | macrophage capping protein | 3 | 50 |
| sw:COF1_HUMAN | P23528 | cofilin, non-muscle isoform (p18). | <2 | 21 |
| sw:DEST_HUMAN | P18282 | destrin (actin-depolymerizing | 2 | 57 |
| | | factor) (adf). | | |
| sw:DYI2_HUMAN | Q13409 | dynein intermediate chain | 2 | 23 |
| | | 2(fragment). | | |
| sw:GELS_HUMAN | P06396 | gelsolin precursor | 3 | 3 |
| sw:K1CS_HUMAN* | P08727 | keratin, type I cytoskeletal 19 | 4 | 33 |
| sw:K2C7_HUMAN | P08729 | keratin, type ii cytoskeletal 7 | 5 | 52 |
| sw:MLEN_HUMAN | P16475 | myosin light chain alkali | 3 | 39 |
| sw:PLSL_HUMAN | P13796 | 1-plastin (lymphocyte cytosolic | <2 | 26 |
| | | protein 1) | | |
| sw:TAGL_HUMAN | P37802 | transgelin (22 kda actin-binding | 2 | 10 |
| | | protein). | | |
| sw:TBA4_HUMAN | P05215 | tubulin alpha-4 chain. | 2 | 8 |
| proteolysis and | | | | |
| peptidolysis | | | | |
| sw:ACY1_HUMAN* | Q03154 | aminoacylase-1 | 5 | 14 |
| sw:AMPL_HUMAN | P28838 | cytosol aminopeptidase | 3 | 32 |
| sw:CATD_HUMAN | P07339 | cathepsin d precursor. | <2 | 1 |
| sw:CFAB_HUMAN | P00751 | complement factor b precursor | 5 | 4 |
| sw:EL3A_HUMAN* | P09093 | elastase iiia precursor | 4 | 35 |
| chaperon | | | | |
| sw:APE_HUMAN | P02649 | apolipoprotein e precursor (apo-e). | <2 | 59 |
| sw:CALX_HUMAN | P27824 | calnexin precursor(p90) | 3 | 40 |
| sw:CYPH_HUMAN | P05092 | peptidyl-prolyl cis-trans isomerase a | <2 | 22 |
| sw:TCPG_HUMAN | P49368 | t-complex protein 1, gamma | 2 | 45 |
| | | subunit | | |
| sw:CBP2_HUMAN | P50454 | human. Collagen-binding protein 2 | 2 | 20 |
| | | precurso | | |
| tr_hum:Q96C61 | Q96C61 | hypothetical 88.6 kda protein | 5 | 60 |
| oxidoreductase | | | | |
| sw:DLDH_HUMAN | P09622 | dihydrolipoamide dehydrogenase | 2 | 36 |
| sw:IDHC_HUMAN* | O75874 | isocitrate dehydrogenase [nadp] | 2 | 2 |
| | | cytoplasmic | | |
| sw:NUAM_HUMAN | P28331 | nadh-ubiquinone oxidoreductase 75 kda | 2 | 42 |
| | | subunit | | |
| metabolism of | | | | |
| biological process | | | | |
| sw:ALDX_HUMAN | P14550 | alcohol dehydrogenase [nadp+] | 4 | 34 |
| sw:ECH1_HUMAN | Q13011 | delta3,5-delta2,4-dienoyl-coa | 2 | 24 |
| | | isomerase | | |
| sw:ECHM_HUMAN | P30084 | enoyl-coa hydratase, mitochondrial | 3 | 37 |
| | | precursor | | |
| sw:IPYR_HUMAN | Q15181 | inorganic pyrophosphatase | <2 | 16 |
| sw:TYPH_HUMAN | P19971 | thymidine phosphorylase precursor | 5 | 31 |
| SW:ENOA_HUMAN | P06733 | human alpha enolase | 2 | 61 |
| sw:ENOL_HUMAN | Q05524 | alpha enolase, lung specific | 2 | 13 |
| sw:SYW_HUMAN | P23381 | tryptophanyl-trna synthetase | 2 | 62 |
| heat shock protein | | | | |
| sw:HSBX_HUMAN | O14558 | heat-shock 20 kda like-protein p20. | 2 | 38 |
| signaling | | | | |
| sw:GBLP_HUMAN | P25388 | guanine nucleotide-binding protein | 2 | 47 |
| | | beta subunit-like protein 12.3 | | |
| sw:GDIR_HUMAN | P52565 | rho gdp-dissociation inhibitor 1 | <2 | 55 |
| sw:IQG1_HUMAN | P46940 | ras gtpase-activating-like protein | 2 | 25 |
| | | iqgap1 | | |
| sw:PBEF_HUMAN | P43490 | pre-b cell enhancing factor | 2 | 43 |
| | | precursor. | | |
| sw:RAN_HUMAN | P17080 | gtp-binding nuclear protein ran | 3 | 27 |
| immune response | | | | |
| sw:KAC_HUMAN | P01834 | ig kappa chain c region. | 2 | 63 |
| sw:MA32_HUMAN | Q07021 | pre-mrna splicing factor sf2, p32 | <2 | 41 |
| | | subunit. | | |
| inflammatory reponse | | | | |
| sw:ANX1_HUMAN | P04083 | annexin I (lipocortin i) | 4 | 51 |
| sw:LEG3_HUMAN | P17931 | galectin-3 | 2 | 17 |
| sw:S109_HUMAN* | P06702 | calgranulin b (mrp-14) | <2 | 49 |
| muscle development | | | | |
| sw:TPM1_HUMAN | P09493 | tropomyosin 1 alpha chain | <2 | 30 |
| sw:TPM4_HUMAN | P07226 | tropomyosin alpha 4 chain | <2 | 11 |
| sw:MLRN_HUMAN | P24844 | myosin regulatory light chain 2 | 2 | 48 |
| transport of | | | | |
| biological process | | | | |
| humangp:CHR2-Q15092 | Q15092 | transmembrane protein. | 2 | 64 |
| sw:POR2_HUMAN | P45880 | voltage-dependent anion-selective | <2 | 18 |
| | | channel protein 2 | | |
| sw:RET1_HUMAN | P09455 | retinol-binding protein I | 2 | 44 |
| RNA processing | | | | |
| humangp:CHR20-Q9P2E9 | O75300 | ribosome binding protein 1 | <2 | 65 |
| | | (kiaa1398 protein). | | |
| sw:RINI_HUMAN | P13489 | placental ribonuclease inhibitor | <2 | 46 |
| sw:ROK_HUMAN | Q07244 | heterogeneous nuclear | 2 | 28 |
| | | ribonucleoprotein k | | |
| blood coagulation | | | | |
| sw:FIBG_HUMAN | P02679 | fibrinogen gamma chain precursor | 3 | 66 |
| sw:THRB_HUMAN | P00734 | prothrombin precursor | <2 | 67 |
| Anti-apoptosis | | | | |
| sw:TCTP_HUMAN | P13693 | translationally controlled tumor | <2 | 29 |
| | | protein (p23) | | |
| cell adhesion | | | | |
| humangp:CHR13-Q15063 | Q15063 | osteoblast specific factor 2 | 2 | 53 |
| | | precursor | | |
| sw:BGH3_HUMAN | Q15582 | transforming growth factor-beta | 5 | 6 |
| | | induced protein | | |
| other | | | | |
| sw:TGLC_HUMAN | P21980 | tissue transglutaminase | 2 | 54 |
| sw:KPY1_HUMAN | P14618 | Human pyruvate kinase, cytosolic | 3 | 68 |
| | | thyroid hormone-binding protein | | |
| humangp:CHR19-Q96D15 | Q96D15 | Reticulocalbin 3 precursor. | 2 | 69 |

We claim:

1. A marker for diagnosis of pancreatic cancer comprising at least one polypeptide selected from the group consisting of the polypeptides having SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, and 55.

2. The marker of claim 1 wherein the group from which at least one polypeptide is selected consists of the polypeptides having SEQ ID NO: 1, 2, 3, 4, 5, 6, 7, 8, 9, 10, 11, 12, 13, 14, 15, 16, 17, 18, 19, 20, 21, 22, 23, 24, 26, 27, 28, 29, 30, 31, 32, 33, 34, 35, 36, 37, 38, 39, 40, 41, 42, 43, 44, 45, 46, 47, 48, and 49.

3. An in vitro method for the diagnosis of pancreatic cancer and/or the susceptibility to pancreatic cancer comprising the steps of
a) obtaining a biological sample; and
b) detecting and/or measuring the increase of the marker of claim 1.

4. The in vitro method of claim 3, wherein the marker comprises at least two polypeptides.

5. The in vitro method of claim 3 wherein said biological sample is derived from the group consisting of serum, plasma, pancreatic juice and cells of pancreatic tissue.

6. An in vitro method for the diagnosis of pancreatic cancer and/or the susceptibility to pancreatic cancer comprising the steps of
a) obtaining a biological sample; and
b) detecting and/or measuring the increase of at least one nucleic acid coding for the marker of claim 1.

7. The in vitro method of claim 6, wherein said nucleic acid molecule is RNA or DNA.

8. The in vitro method of claim 7, wherein said DNA is a cDNA.

9. The in vitro method of claim 6, wherein the expression levels of at least one of said nucleic acids in an individual suspected to suffer from pancreatic cancer and/or to be susceptible to pancreatic cancer is compared to the expression levels of the same nucleic acids in a healthy individual.

10. The in vitro method of claim 3, wherein the expression level of said marker in an individual suspected to suffer from pancreatic cancer and/or to be susceptible to pancreatic cancer is compared to the expression levels of the same marker in a healthy individual.

11. The in vitro method of claim 6, wherein the expression level of said marker in an individual suspected to suffer from pancreatic cancer and/or to be susceptible to pancreatic cancer is compared to the expression levels of the same marker in a healthy individual.

12. The in vitro method of claim 11, wherein an increase of the expression levels of said marker is indicative of pancreatic cancer or the susceptibility to pancreatic cancer.

13. A screening method for identifying and/or obtaining a compound which interacts with at least one polypeptide having a SEQ ID NO: selected from the group consisting of SEQ ID NO: 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, and 55, whose expression is upregulated in pancreatic cancer, comprising the steps of
a) contacting said polypeptide with a compound or a plurality of compounds under conditions which allow interaction of said compound with said polypeptide; and
b) detecting the interaction between said compound or plurality of compounds with said polypeptide.

14. A screening method for identifying and/or obtaining a compound which is an inhibitor or an antagonist of at least one polypeptide having a SEQ ID NO: selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, and 55, whose expression is upregulated in pancreatic cancer, comprising the steps of
a) contacting a said polypeptide with a compound identified and/or obtained by the screening method of claim 13 under conditions which allow interaction of said compound with said polypeptide;
b) determining the activity of said polypeptide;
c) determining the activity of said polypeptide expressed in the host as defined in (a), which has not been contacted with said compound; and
d) quantitatively relating the activity as determined in (b) and (c), wherein a decreased activity determined in (b) in comparison to (c) is indicative for an inhibitor or antagonist.

15. A screening method for identifying and/or obtaining a compound which is an inhibitor of the expression of a polypeptide having a SEQ ID NO: selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, and 55, whose expression is upregulated in pancreatic cancer, comprising the steps of
a) contacting a host which expresses said polypeptide with a compound,
b) determining the expression level and/or activity of said polypeptide;
c) determining the expression level and/or activity of said polypeptide in the host as defined in (a), which has not been contacted with said compound; and
d) quantitatively relating the expression level of said polypeptide as determined in (b) and (c), wherein a decreased expression level determined in (b) in comparison to (c) is indicative for an inhibitor of the expression of said polypeptide.

16. Antibodies against at least one polypeptide having a SEQ ID NO: selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, and 55, or antigen-binding fragments thereof, for the use in an in vitro method for the diagnosis of pancreatic cancer.

17. A kit for the diagnosis of pancreatic cancer comprising one or more of the antibodies, or antigen-binding fragments thereof, of claim 16.

18. A kit for the diagnosis of pancreatic cancer comprising one or more of the nucleic acids coding for the marker of claim 1.

19. A kit for screening of compounds that activate or inhibit any of the polypeptides having a SEQ ID NO: selected from the group consisting of SEQ ID NO: 1, 2, 3, 4, 5, 6, 7, 8, 9, 10, 11, 12, 13, 14, 15, 16, 17, 18, 19, 20,21,22, 23, 24, 25, 26,27, 28, 29, 30, 31, 32, 33, 34, 35, 36, 37, 38, 39, 40, 41, 42, 43, 44, 45, 46, 47, 48, 49, 50, 51, 52, 53, 54, and 55, or stimulate or inhibit the expression of any of said polypeptides.

20. A marker for diagnosis of pancreatic cancer comprising at least one polypeptide selected from the group consisting of the polypeptides having SEQ ID NO: 56, 7, 5, 9, 19, 12, 15, 50, 21, 57, 23, 3, 33, 52, 39, 26, 10, 8, 14, 32, 1, 4, 35, 59, 40, 22, 45, 20, 60, 36, 2, 42, 34, 24, 37, 16, 31, 61, 13, 62, 38, 47, 55, 25, 43, 27, 63, 41, 51, 17, 49, 30, 11, 48, 64, 18, 44, 65, 46, 28, 66, 67, 29, 53, 6, 54, 68 and 69.

21. The marker of claim 20, wherein said at least one polypeptide does not include SEQ ID NO.s 25, 50, 51, 52, 53, 54 and 55.

22. The marker according to claim 20, additionally comprising at least one of the polypeptides having SEQ ID NO: 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 and 110.

23. An in vitro method for the diagnosis of pancreatic cancer and/or the susceptibility to pancreatic cancer comprising the steps of
a) obtaining a biological sample; and
b) detecting and/or measuring the increase of at least one of the polypeptides listed having SEQ ID NO: 56, 7, 5, 9, 19, 12, 15, 50, 21, 57, 23, 3, 33, 52, 39, 26, 10, 8, 14, 32, 1, 4, 35, 59, 40, 22, 45, 20, 60, 36, 2, 42, 34, 24, 37, 16, 31, 61, 13, 62, 38, 47, 55, 25, 43, 27, 63, 41, 51, 17, 49, 30, 11, 48, 64, 18, 44, 65,46, 28, 66, 67, 29, 53, 6, 54, 68 and 69.

24. The in vitro method of claim 23, additionally comprising the step of detecting and/or measuring the decrease of at least one of the polypeptides having SEQ ID NO: 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 and 110.

25. The in vitro method of claim 23, wherein said at least one polypeptide does not include SEQ ID NO.s 25, 50, 51, 52, 53, 54 and 55.

26. The in vitro method of claim 23, wherein said biological sample is derived from the group consisting of serum, plasma, pancreatic juice and cells of pancreatic tissue.

27. An in vitro method for the diagnosis of pancreatic cancer and/or the susceptibility to pancreatic cancer comprising the steps of
a) obtaining a biological sample; and
b) detecting and/or measuring the increase of at least one nucleic acid coding for the marker of claim 23.

28. The in vitro method of claim 27, wherein said nucleic acid molecule is RNA or DNA.

29. The in vitro method of claim 28, wherein said DNA is a cDNA.

30. The in vitro method of claim 27, wherein the expression levels of at least one of said nucleic acids in an individual suspected to suffer from pancreatic cancer and/or to be susceptible to pancreatic cancer is compared to the expression levels of the same nucleic acids in a healthy individual.

31. The in vitro method of claim 27, wherein the expression level of said marker in an individual suspected to suffer from pancreatic cancer and/or to be susceptible to pancreatic cancer is compared to the expression levels of the same marker in a healthy individual.

32. The in vitro method of claim 31, wherein an increase of the expression levels of said marker is indicative of pancreatic cancer or the susceptibility to pancreatic cancer.

33. A screening method for identifying and/or obtaining a compound which interacts with a polypeptide selected from the group consisting of the polypeptides having SEQ ID NO: 56, 7, 5, 9, 19, 12, 15, 50, 21, 57, 23, 3, 33, 52, 39, 26, 10, 8, 14, 32, 1, 4, 35, 59, 40, 22, 45, 20, 60, 36, 2, 42, 34, 24, 37, 16, 31, 61, 13, 62, 38, 47, 55, 25, 43, 27, 63, 41, 51, 17, 49, 30, 11, 48, 64, 18, 44, 65, 46, 28, 66, 67, 29, 53, 6, 54, 68 and 69, whose expression is upregulated in pancreatic cancer, comprising the steps of
a) contacting said polypeptide with a compound or a plurality of compounds under conditions which allow interaction of said compound with said polypeptide; and
b) detecting the interaction between said compound or plurality of compounds with said polypeptide.

34. A screening method for identifying and/or obtaining a compound which is an inhibitor or an antagonist of a polypeptide listed in table 6 whose expression is upregulated in pancreatic cancer, comprising the steps of
a) contacting said polypeptide with a compound identified and/or obtained by the screening method of claim 33 under conditions which allow interaction of said compound with said polypeptide;
b) determining the activity of said polypeptide;
c) determining the activity of said polypeptide expressed in the host as defined in (a), which has not been contacted with said compound; and
d) quantitatively relating the activity as determined in (b) and (c), wherein a decreased activity determined in (b) in comparison to (c) is indicative for an inhibitor or antagonist.

35. A screening method for identifying and/or obtaining a compound which is an inhibitor of the expression of a polypeptide selected from the group consisting of the polypeptides having SEQ ID NO: 56, 7, 5, 9, 19, 12, 15, 50, 21, 57, 23, 3, 33, 52, 39, 26, 10, 8, 14, 32, 1, 4, 35, 59, 40, 22, 45, 20, 60, 36, 2, 42, 34, 24, 37, 16, 31, 61, 13, 62, 38, 47, 55, 25, 43, 27, 63, 41, 51, 17, 49, 30, 11, 48, 64, 18, 44, 65, 46, 28, 66, 67, 29, 53, 6, 54, 68 and 69, whose expression is upregulated in pancreatic cancer, comprising the steps of
a) contacting a host which expresses said polypeptide with a compound,
b) determining the expression level and/or activity of said polypeptide;
c) determining the expression level and/or activity of said polypeptide in the host as defined in (a), which has not been contacted with said compound; and
d) quantitatively relating the expression level of said polypeptide as determined in (b) and (c), wherein a decreased expression level determined in (b) in comparison to (c) is indicative for an inhibitor of the expression of said polypeptide.

36. Antibodies against the proteins having SEQ ID NO: 56, 7, 5, 9, 19, 12, 15, 50, 21, 57, 23, 3, 33, 52, 39, 26, 10, 8, 14, 32, 1, 4, 35, 59, 40, 22, 45, 20, 60, 36, 2,42, 34, 24, 37, 16, 31, 61, 13, 62, 38,47, 55, 25, 43, 27, 63, 41, 51, 17, 49, 30, 11, 48, 64, 18, 44, 65, 46, 28, 66, 67, 29, 53, 6, 54, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 and 110, or antigen-binding fragments thereof, for the use in an in vitro method for the diagnosis of pancreatic cancer.

37. A kit for the diagnosis of pancreatic cancer comprising one or more of the antibodies, or antigen-binding fragments thereof, of claim 36.

38. A kit for the diagnosis of pancreatic cancer comprising one or more of the nucleic acids coding for the marker of claim 20.

39. A kit for screening of compounds that activate or inhibit any of the polypeptides having SEQ ID NO: 56, 7, 5, 9, 19, 12, 15, 50, 21, 57, 23, 3, 33, 52, 39, 26, 10, 8, 14, 32, 1, 4, 35, 59, 40, 22, 45, 20, 60, 36, 2, 42, 34, 24, 37, 16, 31, 61, 13, 62, 38, 47, 55, 25, 43, 27, 63, 41, 51, 17, 49, 30, 11, 48, 64, 18, 44, 65, 46, 28, 66, 67, 29, 53, 6, 54, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 and 110, or stimulate or inhibit the expression of any of said polypeptides.

40. The kit of claim 39, wherein said polypeptides are the polypeptides selected from the group consisting of SEQ ID NO: 56, 7, 5, 9, 19, 12, 15, 50, 21, 57, 23, 3, 33, 52, 39, 26, 10, 8, 14, 32, 1, 4, 35, 59, 40, 22, 45, 20, 60, 36, 2, 42, 34, 24, 37, 16, 31, 61, 13, 62, 38, 47, 55, 25, 43, 27, 63, 41, 51, 17, 49, 30, 11, 48, 64, 18, 44, 65, 46, 28, 66, 67, 29, 53, 6, 54, 68 and 69.
